(19) United States
(12) Patent Application Publication
(10) Pub. No.: US 2005/0131467 A1
(43) Pub. Date: Jun. 16, 2005

(54) Method and apparatus for electrical stimulation therapy for at least one of atrial fibrillation, congestive heart failure, inappropriate sinus tachycardia, and refractory hypertension

(76) Inventor: Birinder R. Boveja, Milwaukee, WI (US)

Correspondence Address: BIRINDER R. BOVEJA & ANGELY WIDHANY, P. O. BOX 210095, MILWAUKEE, WI, 53221, US

(21) Appl. No.: 10/700,040

(22) Filed: Nov. 2, 2003

(51) Int. Cl. A61N 1/362

(52) U.S. Cl. 607/9; 607/5

(57) ABSTRACT

A method and system for providing pulsed electrical stimulation to vagus nerve(s) for providing therapy for cardiovascular disorders such as atrial fibrillation, congestive heart failure, inappropriate sinus tachycardia, and refractory hypertension. Method and system comprising implantable and external components. Power source for the stimulation pulses may be external or implantable. In the preferred embodiment, the implanted stimulator can function as a stimulus-receiver for use with an external stimulator, or may function as a programmable implanted pulse generator (IPG). The external stimulator having capability for networking with other computers, whereby the programs of external stimulator may be remotely controlled over a wide area network. The programmer also having means for networking to exchange data with remote computers.

This application is related to U.S. patent application Ser. No. 09/837,512 filed Apr. 19, 2001.

### FIELD OF INVENTION

This invention relates generally to medical device system for therapy of cardiovascular disorders, more specifically to provide therapy for certain cardiovascular disorders by pulsed electrical stimulation/neuromodulation of vagus nerve(s).

### BACKGROUND

U.S. patent application Ser. No. 09/837,512, assigned to the same assignee as the current patent application, is entitled “Apparatus and method for electrical stimulation adjunct (add-on) therapy of atrial fibrillation, inappropriate sinus tachycardia, and refractory hypertension with an external stimulator. The methods disclosed in the above patent application can be practiced with a stimulator which may have a variety of power sources, as described in this disclosure. Correspondingly, the methods of providing therapy for diabetes, obesity and compulsive eating disorders, and coma as disclosed in applicant's related patent application's (Ser. No. 09/837,662 which is now U.S. Pat. No. 6,654,102, Ser. No. 09/837,660 which is now U.S. Pat. No. 6,615,081, Ser. No. 09/837,661 which is now U.S. Pat. No. 6,611,715, Ser. No. 09/178,060 which is now U.S. Pat. No. 6,205,359, and Ser. No. 09/727,570 which is now U.S. Pat. No. 6,356,788) can also be practiced with a stimulator which may have variety of power sources as disclosed herein. The disclosures of the above patent applications being incorporated herein in their entirety.

The method and system of the current invention utilizes an implantable pulse generator, or an external stimulator in conjunction with an implanted stimulus-receiver to provide therapy, or alleviation of symptoms for certain cardiovascular disorders, such as atrial fibrillation, congestive heart failure (CHF), inappropriate sinus tachycardia, and refractory hypertension. The method and system of this invention delivers pre-determined electrical pulses for neuromodulation of the vagus nerve(s) with an implanted pulse generator (IPG), or an external stimulator along with an implanted stimulus-receiver. The predetermined stimulation pulses comprise unique combinations of pulse amplitude, pulse width, frequency of pulses, on-time and off-time. In one embodiment, the system also contains a telecommunications module within the external stimulator. In such an embodiment, the external stimulator can be controlled remotely, via wireless communication.

### Nervous Control of the Heart

The principle control of heart rate is via the autonomic nervous system. Normally, the average heart rate is approximately 70 beats per minute at rest. During sleep the heart rate diminishes by 10 to 20 beats per minute, but during emotional excitement or muscular activity it may accelerate to rates considerably above 100. In well-trained athletes at rest, the rate is usually only about 50 beats per minute.

The sinoatrial (SA) node 82 of the heart (shown in FIG. 1) is usually under the tonic influence of both divisions of the autonomic nervous system. The sympathetic system enhances automaticity, by increasing the phase 4 depolarization of the pacemaker cells in the sinus node 82, as is shown in FIG. 2A, versus the resting state shown in FIG. 2B. The parasympathetic system inhibits the automaticity (such as with right vagus nerve stimulation). Changes in heart rate (HR) usually involve a reciprocal action of the two divisions of the autonomic nervous system. Thus an increased heart rate is produced by a diminution of parasympathetic activity and concomitant increase in sympathetic activity, and deceleration is usually achieved by the opposite mechanisms. Under certain conditions the heart rate may change by selective action of just one division of the autonomic nervous system, rather than by reciprocal changes in both divisions.

Ordinarily, during rest parasympathetic influences preponderate over sympathetic effects at the SA node. Abolition of parasympathetic influences by administration of atropine usually increases heart rate substantially, whereas abolition of sympathetic effects by administration of propranolol usually decreases heart rate only slightly. When both divisions of the autonomic nervous system are blocked, the heart rate averages about 100 beats per minute. The rate that prevails after complete autonomic blockade is the intrinsic heart rate.

The cardiac parasympathetic fibers originate in the medulla oblongata (of the brain), shown schematically in FIG. 3, in cells that lie in the dorsal motor nucleus of the vagus or in the nucleus ambiguus. Efferent vagal fibers pass inferiorly through the neck as the cervical vagus nerves, which lie close to the common carotid arteries. They then pass through the mediastinum to synapse with postganglionic cells on the epicardial surface or within the walls of the heart itself (shown schematically in FIG. 4). Most of the cardiac ganglion cells are located near the SA node 82 and atrio-ventricular (AV) 84 conduction tissue. The right and left vagi are distributed differentially to the various cardiac structures. The right vagus nerve affects the SA node 82 predominantly. Stimulation slows SA nodal firing or may even stop it for several seconds. The left vagus nerve mainly inhibits AV conduction tissue, to produce various degrees of AV block. However, the distributions of the efferent vagal fibers overlap, such that left vagal stimulation also depresses the SA node and right vagal stimulation impedes AV conduction.

When the right vagus nerve is stimulated at a constant frequency for several seconds, the heart rate decreases abruptly and attains a steady-state value within one or two cardiac cycles. Also, when stimulation is discontinued, the heart rate returns very quickly to its basal level. The combination of the brief latency and rapid decay of the response (because of the abundance of cholinesterase) provides the opportunity for the vagus nerves to exert a beat by beat control of SA and AV nodal function. The vagal preponderance in the regulation of heart rate is mediated mainly by an effective throttling of the release of norepinephrine form the sympathetic nerve endings by the acetylcholine released form neighboring vagus nerve ending.

The cardiac sympathetic fibers originate in the intermediolateral columns of the upper five or six thoracic and lower one or two cervical segments of the spinal cord. They emerge form the spinal column through the white communication branches and enter the paravertebral chains of ganglia. The preganglionic and postganglionic neurons synapse mainly in the stellate and middle cervical ganglia FIG. 4. The middle cervical ganglia lie close to the vagus nerves in the superior portion of the mediastnum. Sympathetic and parasympathetic fibers then join to form a complex plexus of mixed efferent nerves to the heart.

As with the vagus nerves, the left and right sympathetic fibers are distributed differentially. At the beginning of sympathetic stimulation, the facilitatory effects on the heart attain steady-state values much more slowly than do the inhibitory effects of vagal stimulation

The two arms of the autonomic nervous system, the parasympathetic and sympathetic divisions, generally serve the same visceral organs but cause essentially opposite effects. A dynamic antagonism exists between the two divisions, and fine adjustments are made continuously by both. If one division stimulates certain smooth muscles to contract or a gland to secrete, the other division inhibits that action. Through this process of dual innervation, the two divisions counterbalance each other's activities to keep body systems running smoothly. The sympathetic part mobilizes the body during extreme situations (such as fear, exercise, or rage), whereas the parasympathetic arm allows us to unwind as it performs maintenance activities and conserves body energy.

To elaborate on these functional differences by focusing briefly on situations in which each division is exerting primary control. The parasympathetic division is most active in non-stressful situations. This division, sometimes called the “resting and digesting” system, is chiefly concerned with keeping body energy use as low as possible. Its activity is best illustrated in a person who relaxes after a meal and reads the newspaper. Blood pressure, heart rate, and respiratory rate are regulated at low normal levels, the gastrointestinal tract is actively digesting food and skin is warm (indicating that there is no need to divert blood to skeletal muscles or vital organs). The major portion of the parasympathetic cranial outflow is via the vagus (X) nerves. Between them, the two vagus nerves account for about 90% of all preganglionic parasympathetic fibers in the body. The provide fibers to the neck and contribute to nerve plexus that serve virtually every organ in the thoracic and abdominal cavities. The vagus nerves are able to exert beat-by-beat control of heart rate, whereas the sympathetic nerves are not able to alter cardiac behavior very much within one cardiac cycle.

The sympathetic division is often referred to as the “fight-or-flight” system. Its activity is evident when we are excited or find ourselves in emergency or threatening situation, such as being frightened by street toughs late at night. A pounding heart; rapid deep breathing; cold, sweaty skin; and dilated eye pupils are sure signs of mobilization of the sympathetic nervous system.

As shown in FIG. 3, The cardiovascular (CV) center 222 located in the medullary center in the brain influences and controls cardiovascular functions such as heart rate, contractactility, and blood vessels. The cardiovascular center 222 in the brain 220, receives input from the higher centers in the brain 224 and from receptors 226 such as baroreceptors and propriocepters. The cardiovascular (CV) center 222 of the brain 220 controls the effector organs in the body by increasing the frequency of nerve impulses. The CV center 222 decreases heart rate by parasympathetic stimulation via efferent impulses carried by the 10^{th} cranial nerve or the vagus nerve. The CV center can also increase heart rate and cause vasoconstriction via sympathetic stimulation. Thus, the CV center 222 in the brain 220 exerts its control via the opposing actions of the sympathetic and parasympathetic stimulation.

Further, as shown in FIG. 3 baroreceptors located in the aortic arch 262, and in the carotid sinus 260 send blood pressure information to the cardiovascular (CV) center 222 located in Medulla Oblongata 240 of the brain 220. This information is carried by afferent fibers of Glossopharyngeal Nerve 55 and Vagus Nerve 54.

Additionally of interest to the current patent application, the efferent fibers of the right vagus nerve predominately innervate the sinus node 252 and stimulation of these fibers will be used to control (slow-down) heart. The efferent fibers from the left vagus nerve predominately innervate the A-V node 256 of heart, and efferent stimulation of the left vagus nerve 54 will be used for controlling heart rate as adjunct (add-on) therapy for atrial fibrillation in this invention.

### Atrial Fibrillation

Atrial fibrillation (AF) is both the most common sustained arrhythmia encountered in clinical practice, and the most common arrhythmia-related cause of hospital admission. Health utilization costs related to atrial fibrillation are significant. Estimates indicate that 2.2 million Americans have AF and that 160,000 new cases are diagnosed each year. The incidence is higher in older adults, whose risk for developing AF is associated with advanced age. During atrial fibrillation, the atria of the heart discharge at a rate between 350 and 600 per minute. The ventricular rate during atrial fibrillation is dependent on the conducting ability of the AV node which is itself influenced by the autonomic system. Atrioventricular conduction will be enhanced by sympathetic nervous system activity and depressed by high vagal tone. In patients with normal atrioventricular conduction, the ventricular rate ranges from 100 to 180 beats per minute.

AF is characterized by a rapid, irregular ventricular rate, the irregularity being in rhythm and arterial pulse pressure amplitude. This can occur to such an extent that multiple pulse deficits (absence of an arterial pulse following ventricular excitation) are present. Current therapies are designed to extinguish the fibrillation activity or to control or abolish atrioventricular (AV) conduction.

Thus, the two components of acute management of patients with atrial fibrillation include control of ventricular rate and conversion to sinus rhythm. The traditional first step in acute treatment of patients with symptomatic AF who have a rapid ventricular response is to slow the ventricular rate. The first line of defense is usually drugs such as Digoxin, Metoprolol, Esmolol and verapamil etc. Drugs typically have side effects, and some patients may be refractory to drugs. Non-pharmacologic adjunct therapy such as nerve stimulation offers an alternative mode of therapy.

In a paper published by Van den Berg et al in the Aug. 19, 1997 issue of Circulation, the authors showed that heart rate variability in patients with atrial fibrillation is related to vagal tone. In an abstract published at the American Heart Association meeting, by Tabata et al. from the Cleveland Clinic Foundation, the authors presented the results of heart rate reduction by vagus nerve stimulation on left ventricular systolic function. Their data showed a dramatic decrease in ejection fraction and stroke volume as atrial fibrillation was induced. Then, while still in atrial fibrillation, a return towards baseline of both ejection fraction and stroke volume, with vagus nerve stimulation of the atrio-ventricular (AV) node.

Thus, with the system of the present invention where pulsed electrical stimulation is provided by an external stimulator or an IPG, with turning the stimulation “on”, the symptoms of atrial fibrillation would be alleviated by decreasing the heart rate and increasing the stroke volume and ejection fraction.

### Congestive Heart Failure (CHF)

Congestive heart failure (CHF) is a condition where the pump efficiency (cardiac output) of the heart becomes so low that blood circulation is inadequate to meet tissue needs. Congestive heart failure is usually a progressively worsening condition resulting in weakening of the heart tissue. Approximately five million Americans suffer from CHF with a significant percentage being under the age of 60 years.

Congestive heart failure (CHF) and certain other disorders such as hypertension and diabetes are typically associated with an increased autonomic cardiovascular drive. Treatment strategies for CHF can employ methods to increase the inhibitory or parasympathetic drive. This can be accomplished via appropriate stimulation of the right vagus nerve. Since right vagus nerve stimulation lowers the heart rate, it also lowers the exercise tolerance of the patient.

In articles published in Cardiovascular Research (1994; vol. 28:1774-1779) and Circulation Research (1981; vol. 49: 469-478), medical researchers have shown increases in myocardial capillary supply with chronic bradycardia (slower heart rates), over different species. Heart performance also improved. Further, the increase in myocardial capillarity was directly proportional to the length of time that the bradycardia (slower heart rates) were maintained.

One of the objects of this invention is to increase the parasympathetic tone by stimulating the vagus nerve (predominately the right vagus nerve), thereby slowing the resting heart rate (HR) of the patient to a value below the normal HR. This is performed only at times when the patient does not have high metabolic need, such as at night preferably during sleep. Over a period of time this will stimulate the growth of myocardial capillaries leading to an improvement in heart function of the patient.

### Inappropriate Sinus Tachycardia

Inappropriate Sinus Tachycardia is a clinical syndrome with a relative or absolute increase of heart rate at rest or an exaggerated heart rate response inappropriate to the degree of physical or emotional stress. On the surface electrocardiogram, P-wave morphology during tachycardia is nearly identical to the P-wave morphology during normal sinus rhythm. The clinical manifestations of this syndrome complex are diverse. Young women make up most of the patient population, and clinical symptoms can range from intermittent palpitations to multiple system complaints.

Clinical signs and symptoms associated with inappropriate sinus tachycardia are often refractory to medical therapy with drugs. Drugs, such as β-adrenergic blockers or calcium channel blockers, usually either are not effective in controlling symptoms or are poorly tolerated. It is hypothesized that the inappropriate sinus tachycardia response in these patients is due to underlying autonomic dysregulation. The electrophysiologic findings are consistent with the diagnosis of inappropriate sinus tachycardia in the following circumstances: Gradual increase (warm-up) and decrease (cool-down) in heart rate during initiation and termination of isoproterenol infusion, consistent with an automatic mechanism of sinus node function; Surface P-wave morphology similar to that observed during sinus rhythm; and Earliest endocardial activation along the crista terminalis estimated from fluoroscopic images. Clinically, Inappropriate Sinus Tachycardia is divided into 2 subsets, a) postural orthostatic tachycardia syndrome (POTS), and b) non-postural orthostatic tachycardia syndrome (non-POTS). The second category, non-POTS would be alleviated by decreasing the heart rate by the system and method of the current invention.

### Hypertension

Blood pressure (BP) is the hydrostatic pressure exerted by blood on the walls of a blood vessel. The arterial blood pressure is determined by physical and physiological factors. Mean arterial pressure is the pressure in the large arteries, averaged over time. Systolic and diastolic arterial pressures are then considered as the upper and lower limits of periodic oscillations about this mean pressure. The pressure of the blood in arteries and arterioles reaches a peak, called systolic pressure, with each contraction of the heart and then gradually decreases to a minimum, the diastolic pressure before the next contraction. Blood pressure is always expressed as two figures, for example, 120/80 in healthy young adults, representing respectively the systolic and diastolic pressures in millimeters of mercury (mm Hg).

About 20% of the adult population is afflicted with hypertension, the most common single disorder seen in the office of an internist. It is a major risk factor for coronary artery disease and a common cause of heart failure, kidney failure, stroke, and blindness. For adults over 50 years of age, the diagnosis is usually based on repeated resting levels of greater than 160/95 mm Hg in adults over 50 years of age. It is more common among males than females and far more common among blacks than whites. In refractory hypertension, the BP stays at these levels despite treatment with at least two anti-hypertensive drugs for a period of time that is normally adequate to relieve the symptoms.

There is considerable evidence that the nervous system is much involved in the regulation of arterial pressure. For example, hypertension can be induced in experimental animals by transection of arterial baroceptor nerves, by lesion of the nucleus tractus solitarius (NTS). For refractory hypertension where pharmacologic therapy either is not effective, or is not tolerated because of the side effects of drugs, non-pharmacologic therapy such as afferent nerve stimulation may be another alternative for adjunct (add-on) therapy. The neuromodulation of the vagus nerve is designed to control the patient's blood pressure, in the system and method of this invention.

### Neuromodulation of Nerve Tissue

One of the fundamental features of the nervous system is its ability to generate and conduct electrical impulses. These can take the form of action potentials, which is defined as a single electrical impulse passing down an axon, and is shown schematically in FIG. 5. The top portion of the figure shows conduction over mylinated axon (fiber) and the bottom portion shows conduction over nonmylinated axon (fiber). These electrical signals will travel along the nerve fibers.

The nerve impulse (or action potential) is an “all or nothing” phenomenon. That is to say, once the threshold stimulus intensity is reached an action potential 7 will be generated. This is shown schematically in FIG. 6. The bottom portion of the figure shows a train of action potentials.

Most nerves in the human body are composed of thousands of fibers of different sizes. This is shown schematically in FIG. 7. The different sizes of nerve fibers, which carry signals to and from the brain, are designated by groups A, B, and C. The vagus nerve, for example, may have approximately 100,000 fibers of the three different types, each carrying signals. Each axon or fiber of that nerve conducts only in one direction, in normal circumstances.

In a cross section of peripheral nerve it is seen that the diameter of individual fibers vary substantially. The largest nerve fibers are approximately 20 μm in diameter and are heavily myelinated (i.e., have a myelin sheath, constituting a substance largely composed of fat), whereas the smallest nerve fibers are less than 1 μm in diameter and are unmyelinated. As shown in FIG. 8, when the distal part of a nerve is electrically stimulated, a compound action potential is recorded by an electrode located more proximally. A compound action potential contains several peaks or waves of activity that represent the summated response of multiple fibers having similar conduction velocities. The waves in a compound action potential represent different types of nerve fibers that are classified into corresponding functional categories as shown in the table below,

| | Conduction | Fiber | |
|---|---|---|---|
| Fiber | Velocity | Diameter | |
| Type | (m/sec) | (μm) | Myelination |
| A Fibers | | | |
| Alpha | 70-120 | 12-20 | Yes |
| Beta | 40-70 | 5-12 | Yes |
| Gamma | 10-50 | 3-6 | Yes |
| Delta | 6-30 | 2-5 | Yes |
| B Fibers | 5-15 | <3 | Yes |
| C Fibers | 0.5-2.0 | 0.4-1.2 | No |

The diameters of group A and group B fibers include the thickness of the myelin sheaths. Group A is further subdivided into alpha, beta, gamma, and delta fibers in decreasing order of size. There is some overlapping of the diameters of the A, B, and C groups because physiological properties, especially in the form of the action potential, are taken into consideration when defining the groups. The smallest fibers (group C) are unmyelinated and have the slowest conduction rate, whereas the myelinated fibers of group B and group A exhibit rates of conduction that progressively increase with diameter.

Compared to unmyelinated fibers, myelinated fibers are typically larger, conduct faster, have very low stimulation thresholds, and exhibit a particular strength-duration curve or respond to a specific pulse width versus amplitude for stimulation. The A and B fibers can be stimulated with relatively narrow pulse widths, from 50 to 200 microseconds (μs), for example. The A fiber conducts slightly faster than the B fiber and has a slightly lower threshold. The C fibers are very small, conduct electrical signals very slowly, and have high stimulation thresholds typically requiring a wider pulse width (300-1,000 μs) and a higher amplitude for activation. Because of their very slow conduction, C fibers would not be highly responsive to rapid stimulation. Selective stimulation of only A and B fibers is readily accomplished. The requirement of a larger and wider pulse to stimulate the C fibers, however, makes selective stimulation of only C fibers, to the exclusion of the A and B fibers, virtually unachievable inasmuch as the large signal will tend to activate the A and B fibers to some extent as well.

The vagus nerve is composed of somatic and visceral afferents and efferents. Usually, nerve stimulation activates signals in both directions (bi-directionally). It is possible however, through the use of special electrodes and waveforms, to selectively stimulate a nerve in one direction only (unidirectionally). The vast majority of vagus nerve fibers are C fibers, and a majority are visceral afferents having cell bodies lying in masses or ganglia in the skull. The central projections terminate largely in the nucleus of the solitary tract, which sends fibers to various regions of the brain (e.g., the thalamus, hypothalamus and amygdala).

Vagus nerve stimulation can be a means of directly affecting central function. As shown in FIG. 9, cranial nerves have both afferent pathway 19 (inward conducting nerve fibers which convey impulses toward the brain) and efferent pathway 21 (outward conducting nerve fibers which convey impulses to an effector). The vagus nerve 54 is composed of 80% afferent sensory fibers carrying information to the brain from the head, neck, thorax, and abdomen. The sensory afferent cell bodies of the vagus reside in the nodose ganglion and relay information to the nucleus tractus solitarius (NTS).

FIG. 10 shows the nerve fibers traveling through the spinothalamic tract to the brain. The afferent fibers project primarily to the nucleus of the solitary tract (shown schematically in FIG. 11) which extends throughout the length of the medulla oblongata. A small number of fibers pass directly to the spinal trigeminal nucleus and the reticular formation. As shown in FIG. 11, the nucleus of the solitary tract has widespread projection to cerebral cortex, basal forebrain, thalamus, hypothalamus, amygdala, hippocampus, dorsal raphe, and cerebellum.

In summary, neuromodulation of the vagal nerve fibers exert their influence on refractory hypertension via Afferent stimulation. And, neuromodulation of the vagal nerve fibers exert their influence on atrial fibrillation and in Inappropriate Sinus Tachycardia Syndrome via Efferent stimulation of the left and right vagus nerve respectively.

### PRIOR ART

U.S. Pat. No. 5,707,400 (Terry et al.) is generally directed to using an implantable device like a “cardiac pacemaker” for treating refractory hypertension by nerve stimulation. The implanted pulse generator of this patent is programmed by an external personnel computer based programmer with a modified wand.

U.S. Pat. No. 5,690,681 (Geddes et al.) is directed to a closed-loop implanted vagal stimulation apparatus for control of ventricular rate during atrial fibrillation. In this patent, implanted cardiac leads, and implanted pulse generator are used for sensing signals from atrial and ventricular electrograms and an adaptive control system (controller) is used for closing the loop for output stimulation to the vagus nerve. In the current patent application, the patient acts as the feedback loop.

U.S. Pat. No. 5,916,239 (Geddes et al.) is directed to apparatus and method for automatically and continuously adjusting the frequency of nerve stimulator as a function of signals obtained via atrial and ventricular electrograms.

U.S. Pat. No. 5,700,282 (Zabara) is directed to simultaneously stimulating vagus efferents and cardiac sympathetic nerve efferents. The rationale being to employ the brain's natural mechanisms for heart rhythm control.

U.S. Pat. No. 5,522,854 (Ideker et al.) is generally directed to monitoring parasympathetic and sympathetic nerve activity and stimulating the afferent nerves with an implanted device, with the goal of preventing arryhthmias.

U.S. Pat. No. 5,199,428 (Obel et al.) is directed to an implantable electrical nerve stimulator/pacemaker for decreasing cardiac workload for myocardial ischemia. The methodology involves stimulating the carotid sinus nerves or the stellate ganglion.

U.S. Pat. No. 5,330,507 (Schwartz) is generally directed to stimulating right or left vagus nerve with an implanted device which is an extension of a dual chamber cardiac pacemaker.

U.S. Pat. Nos. 6,473,644 B1 (Terry, Jr. et al.) and 6,622,041 B2 (Terry, Jr. et al.) are generally directed to treating patients suffering from heart failure to increase cardiac output, using Neurocybernetic Prosthesis (NCP).

U.S. Pat. No. 4,573,481 (Bullara) is directed to an implantable helical electrode assembly configured to fit around a nerve. The individual flexible ribbon electrodes are each partially embedded in a portion of the peripheral surface of a helically formed dielectric support matrix.

U.S. Pat. No. 3,760,812 (Timm et al.) discloses nerve stimulation electrodes that include a pair of parallel spaced apart helically wound conductors maintained in this configuration.

U.S. Pat. No. 4,979,511 (Terry) discloses a flexible, helical electrode structure with an improved connector for attaching the lead wires to the nerve bundle to minimize damage.

The method and system of the current invention offers many advantages over the prior art for delivering electrical stimulation neuromodulation therapy for atrial fibrillation, congestive heart failure (CHF), inappropriate sinus tachycardia, and refractory hypertension. Further, the programmability of the external stimulator can be controlled remotely, via the wireless medium, as described in a co-pending application.

### SUMMARY OF THE INVENTION

A method and system to selectively stimulate vagus nerve(s) fibers utilizing a combination of external and implantable power sources. More specifically a device wherein the patient can selectively alternate between an implanted and external power sources to be able to provide optimal therapy regiment for cardiovascular disorders, comprising atrial fibrillation, congestive heart failure (CHF), inappropriate sinus tachycardia, and refractory hypertension. Furthermore, the programming of the external stimulator can be controlled remotely.

In one aspect of the invention, an external stimulator may be used along with an implanted stimulus-receiver, wherein the implanted stimulus-receiver comprises a high value capacitor for storing charge for up to 24 hours.

In another aspect of the invention, a programmerless implantable pulse generator may be used, wherein a limited number of states can be programmed with a magnet.

In another aspect of the invention, a combination of stimulus-receiver and implanted pulse generator (IPG) can be used; wherein the IPG can be a stand alone or can be used as stimulus-receiver in conjunction with an external stimulator.

In another aspect of the invention, an implanted pulse generator (IPG) comprises a re-charge coil external to the IPG can, wherein the IPG can be re-charged using an external power sourc.

In yet another aspect of the invention, the electrical stimulation system can be remotely interrogated and programmed over wireless wide area network.

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a schematic diagram of the anatomy of the heart showing the SA node and the AV node.

FIGS. 2A and 2B are diagrams showing recordings from the sinus node.

FIG. 3 is a schematic diagram showing inputs and outputs to the cardiovascular center in the brain.

FIG. 4 is a simplified schematic diagram showing nervous control of the heart.

FIG. 5 is a schematic diagram of myelinated and nonmyelinated axon.

FIG. 6 is a schematic diagram of a single nerve impulse and a train of nerve impulses.

FIG. 7 is a diagram of the structure of a peripheral nerve.

FIG. 8 is a diagram showing recordings of compound action potentials.

FIG. 9 is a schematic diagram of brain showing afferent and efferent pathways.

FIG. 10 is a schematic diagram showing pathways along the spinothalamic tract.

FIG. 11 is a schematic diagram showing relationship of Nucleus of the Solitary Track and how it relays information to other parts of the brain.

FIG. 12 is a schematic diagram of a patient with an implanted stimulus-receiver and an external stimulator.

FIG. 13 is a diagram showing the implanted lead-receiver in contact with the vagus nerve at the distal end.

FIG. 14 is a schematic of the passive circuitry in the implanted lead-receiver.

FIG. 15A is a schematic of an alternative embodiment of th implanted lead-receiver.

FIG. 15B is another alternative embodiment of the implanted lead-receiver.

FIG. 16 shows coupling of the external stimulator and the implanted stimulus-receiver.

FIG. 17 is a top-level block diagram of the external stimulator and proximity sensing mechanism.

FIG. 18 is a diagram showing the proximity sensor circuitry.

FIG. 19 shows the pulse train to be transmitted to the vagus nerve.

FIG. 20 shows the ramp-up and ramp-down characteristic of the pulse train.

FIG. 21 is a schematic diagram of the implantable lead.

FIG. 22 is a schematic diagram showing the implantable lead and one form of stimulus-receiver.

FIG. 23 is a schematic block diagram showing a system for neuromodulation of the vagus nerve, with an implanted component which is both RF coupled and contains a capacitor power source.

FIG. 24A is a simplified block diagram showing control of the implantable neurostimulator with a magnet.

FIG. 24B is a schematic diagram showing implementation of a multi-state converter.

FIG. 25 is a schematic diagram depicting digital circuitry for state machine.

FIG. 26 is a simplified block diagram of the implantable pulse generator.

FIGS. 27A and 27B are diagrams showing communication of programmer with the implanted stimulator.

FIGS. 28A and 28B show diagrammatically encoding and decoding of programming pulses.

FIG. 29 is a diagram showing the two modules of the implanted pulse generator (IPG).

FIG. 30 is a schematic and functional block diagram showing the components and their relationships to the implantable pulse generator/stimulus-receiver.

FIGS. 31A, 31B and 31C show output pulses from a comparator when input exceeds a reference voltage.

FIGS. 32A and 32B are simplified block diagrams showing the switching relationships between the inductively coupled and battery powered assemblies of the pulse generator.

FIG. 33 shows details of implanted pulse generator.

FIG. 34 shows details of digital components of the implantable circuitry.

FIG. 35 shows a picture of the combination implantable stimulator.

FIG. 36 shows assembly features of the implantable portion of the system.

FIG. 37 depicts an embodiment where the implantable system is used as an implantable, rechargeable system.

FIG. 38 is an overall schematic diagram of the external stimulator, showing wireless communication.

FIG. 39 is a schematic diagram showing application of Wireless Application Protocol (WAP).

FIG. 40 is a simplified block diagram of the networking interface board.

FIGS. 41A and 41B is a simplified diagram showing communication of modified PDA/phone with an external stimulator via a cellular tower/base station.

### DETAILED DESCRIPTION OF THE INVENTION

The following description is of the current embodiment for carrying out the invention. This description is not to be taken in a limiting sense, but is made merely for the purpose of describing the general principles of the invention. The scope of the invention should be determined with reference to the claims.

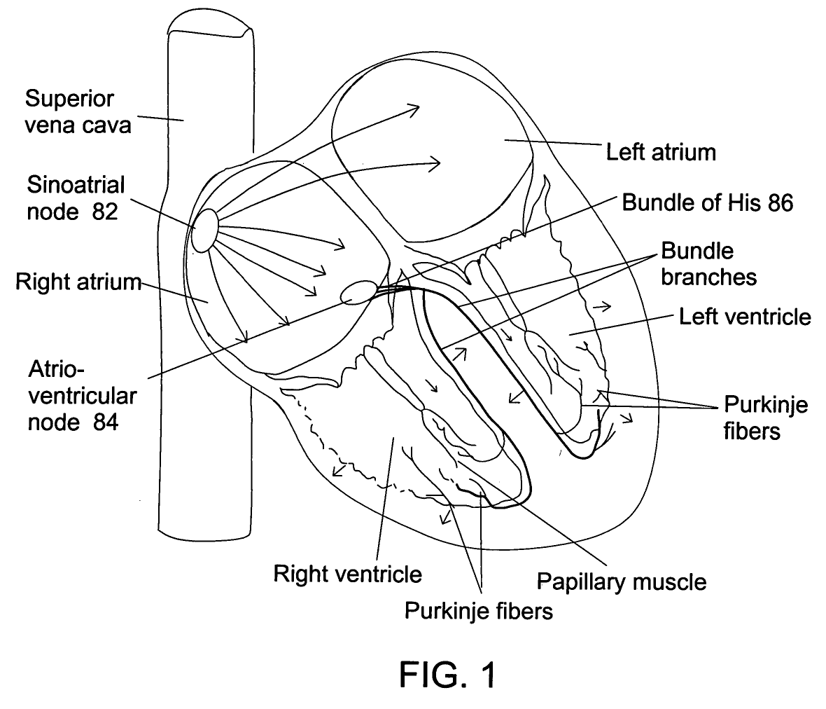

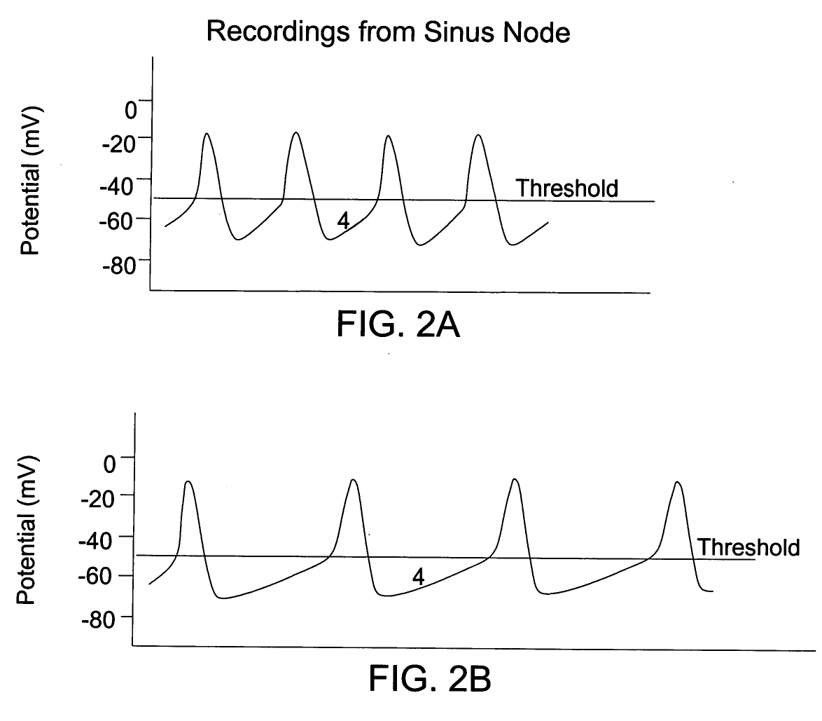

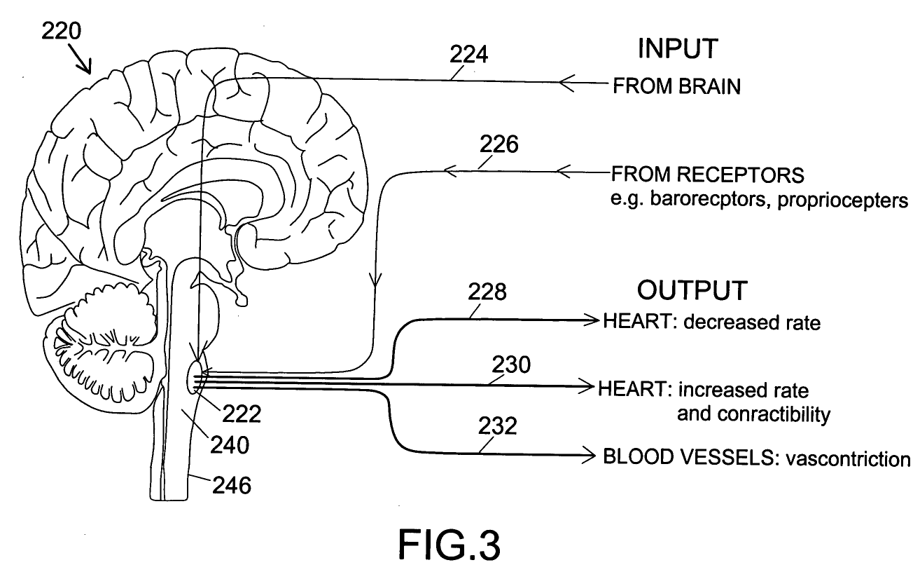

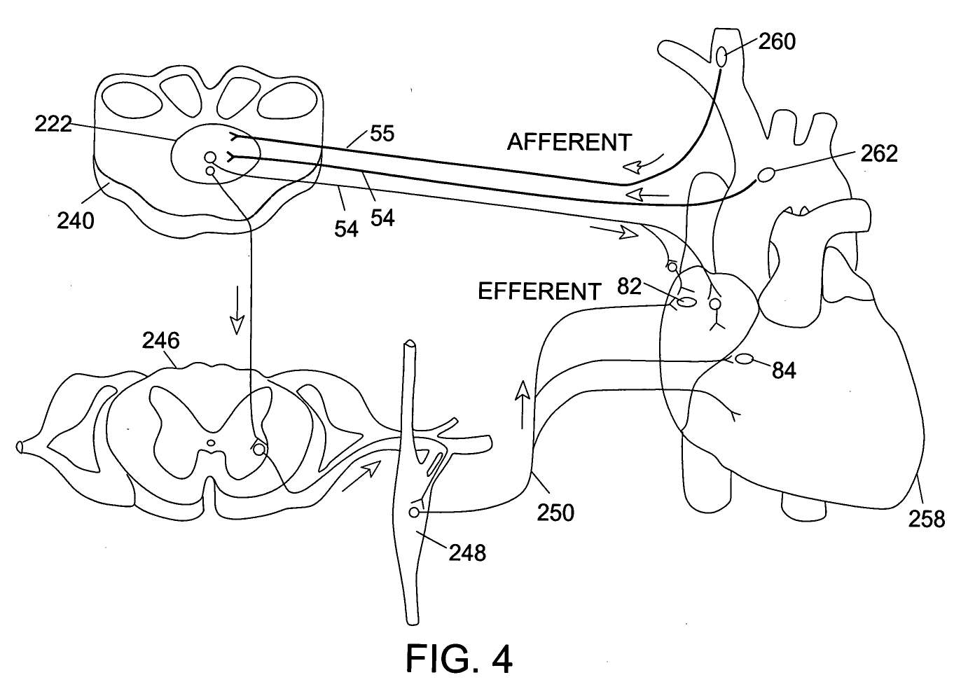

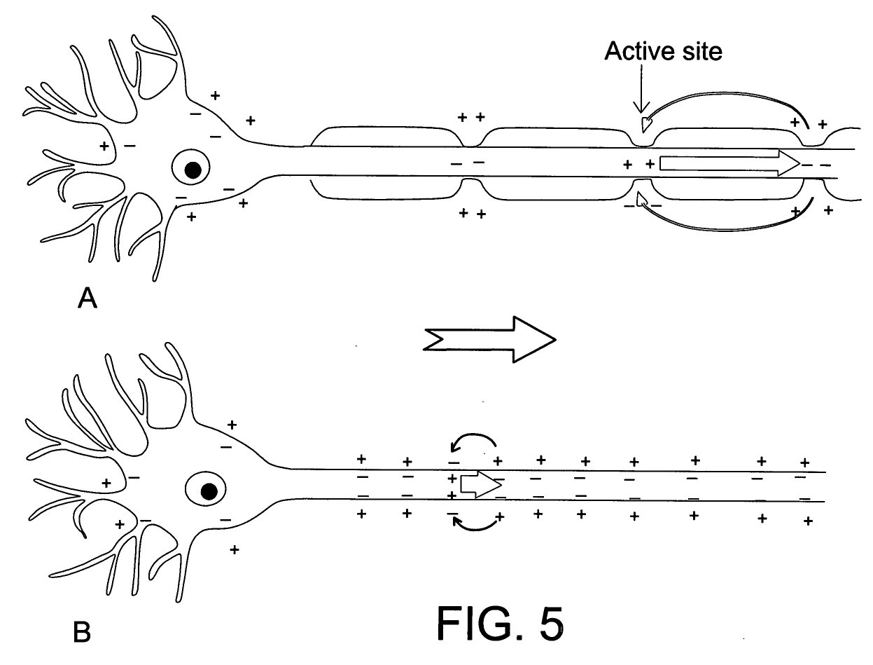

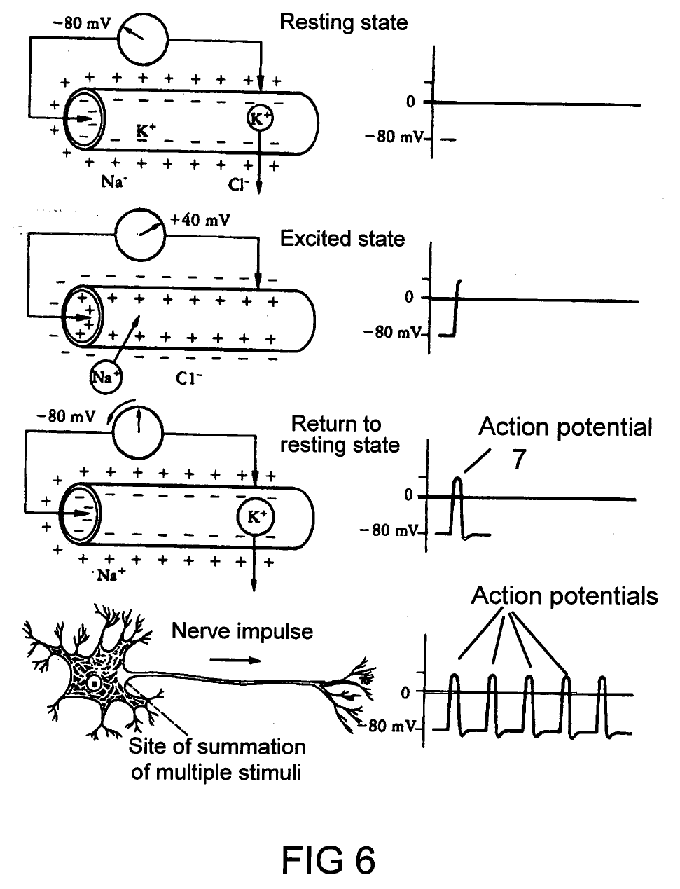

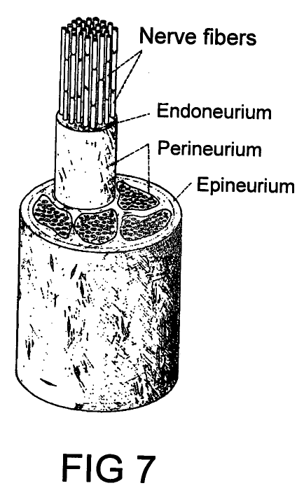

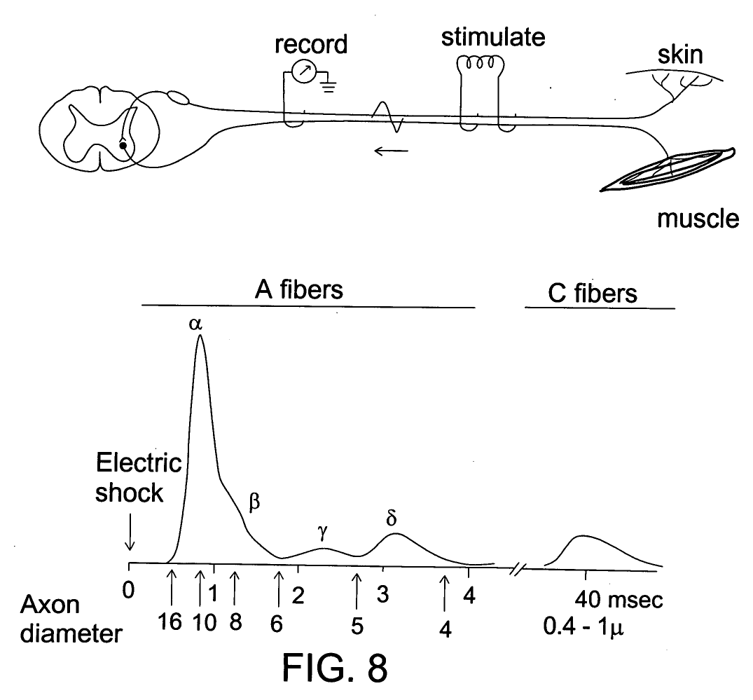

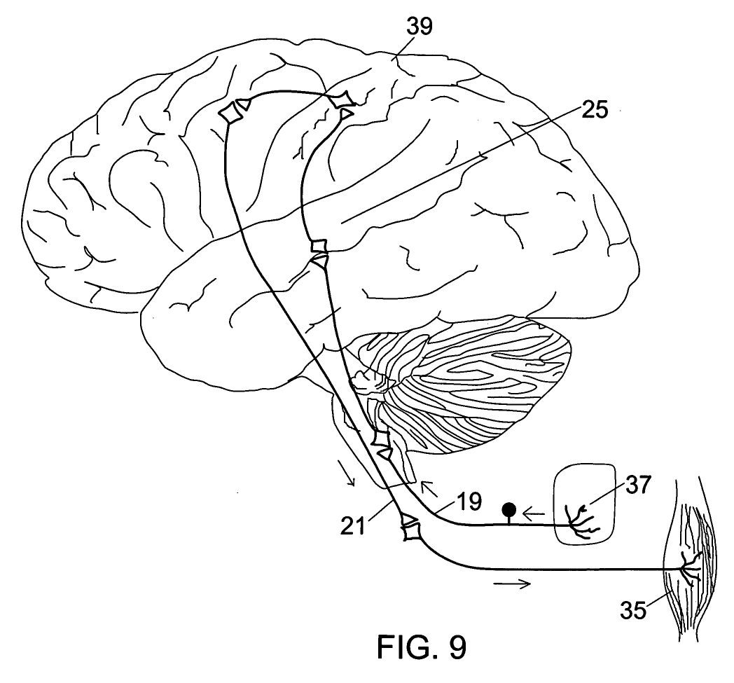

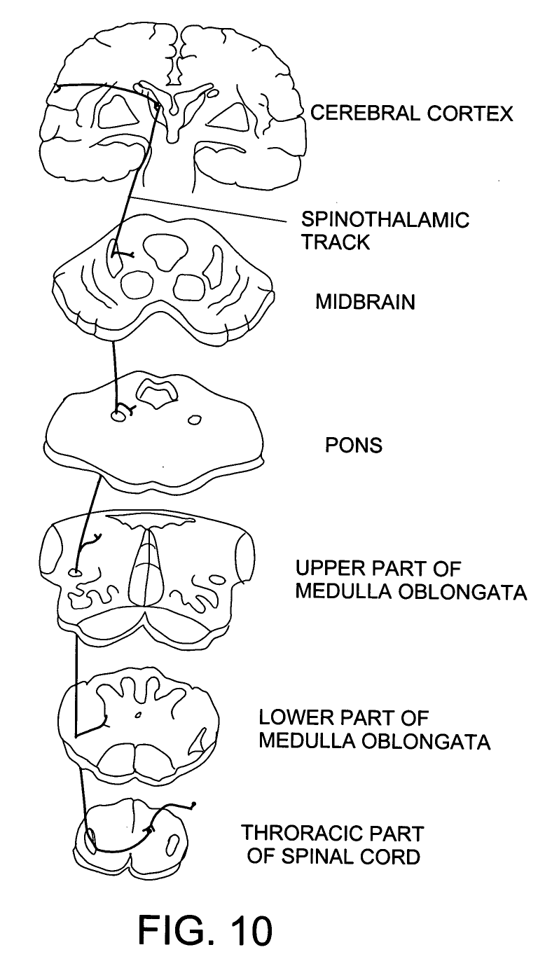

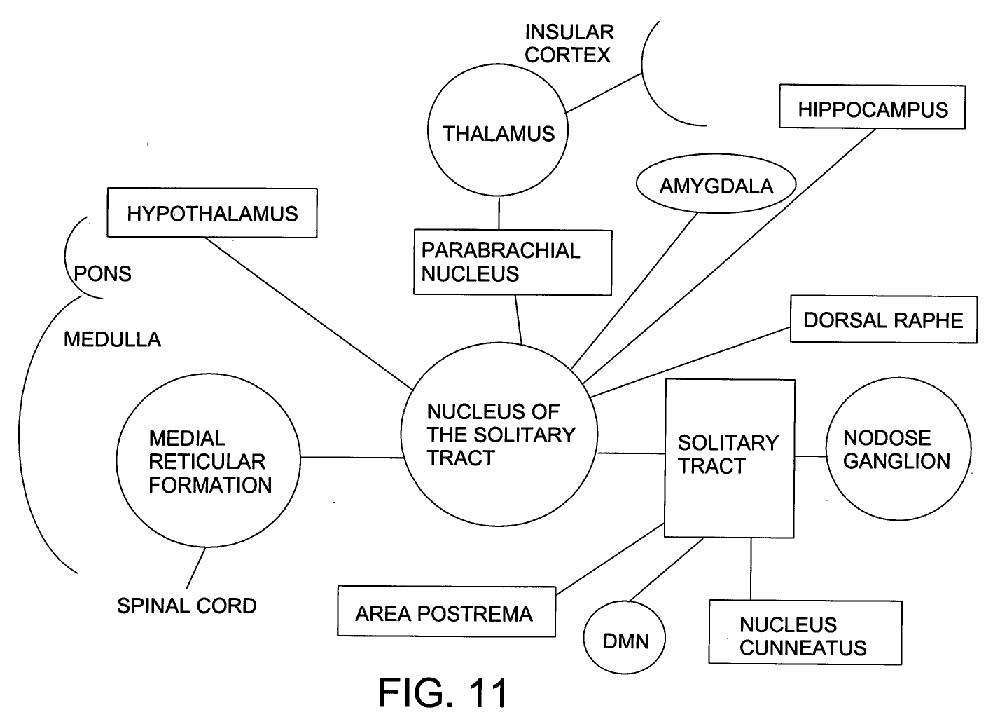

The method and system of neuromodulation therapy of this invention comprises delivering pulsed electrical stimulation to the nerve tissue, such as the vagus nerve, using an implanted pulse generator (IPG) or an external stimulator. The electrical stimulation neuromodulation is to the right vagus nerve to provide therapy for congestive heart failure (CHF), and inappropriate sinus tachycardia, where selective efferent stimulation leads to bradycardia or slowing of the heart rate (HR), due to direct influence on the sino-atrial (SA) node 82 of the heart (FIGS. 1 and 2). The selective stimulation is to the left vagus nerve to provide therapy for atrial fibrillation where efferent stimulation leads to prolongation of conduction time through the atrio-ventricular (AV) node 84 of the heart. And, the selective stimulation is to the left vagus nerve to provide therapy for refractory hypertension, where afferent neuromodulation leads to centrally mediated effects via projections from the medullary centers of the brain, shown in FIG. 11.

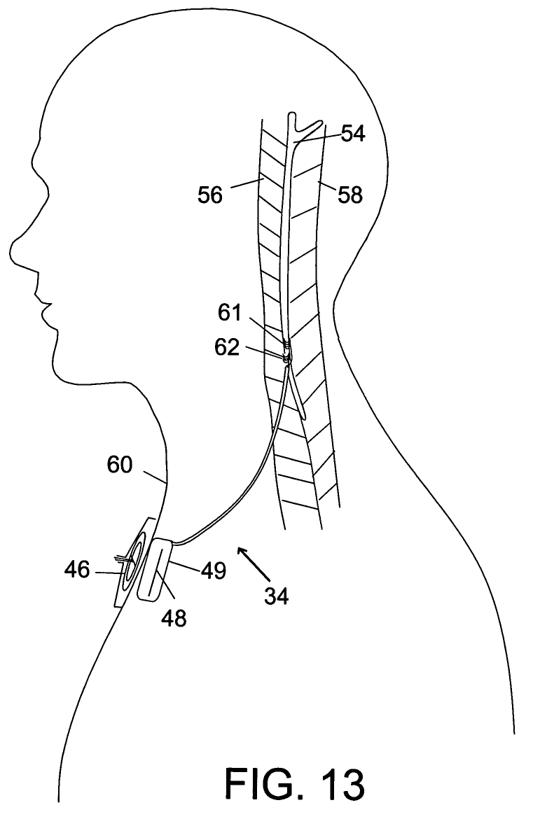

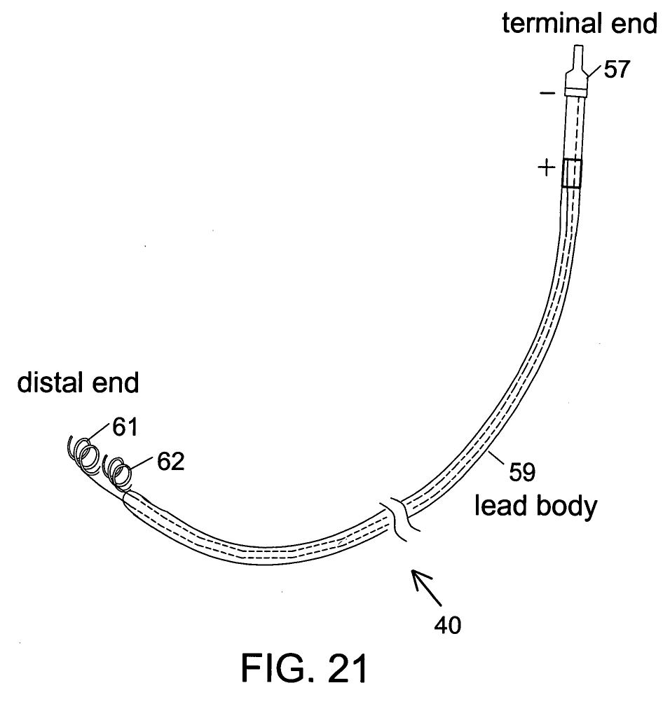

Electrical stimulation neuromodulation pulses to either the right or left vagus nerves are provided via a lead, preferably of the type shown in FIG. 21 (described later). The distal end of the lead has two electrodes 61,62 in direct electrical contact with the appropriate vagus nerve tissue 54. During the surgical implant procedure, the stimulating electrodes 61,62 are tunneled subcutaneously and the spiral shaped electrodes are wrapped around the vagus nerve 54, which is surgically isolated from the carotid artery 56 and jugular vein 58 (FIG. 13). The proximal or terminal end of the lead is connected to pulse generator circuitry. The incisions are surgically closed in the usual manner, and the chronic stimulation process can begin when the tissues are healed from the surgery.

The electrical stimulation system comprises both implanted and external components. The power source may be external, implantable, or a combination device. Some examples of stimulation and power sources that may be used for the practice of this invention include:

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator;

e) a combination implantable device comprising both a stimulus-receiver and a programmable IPG; and

f) an IPG comprising a rechargeable battery.

### Implanted Stimulus-Receiver with an External Stimulator

For an external power source, a passive implanted stimulus-receiver may be used. Such a system is described in the parent application Ser. No. 09/837,512 and mentioned here for convenience.

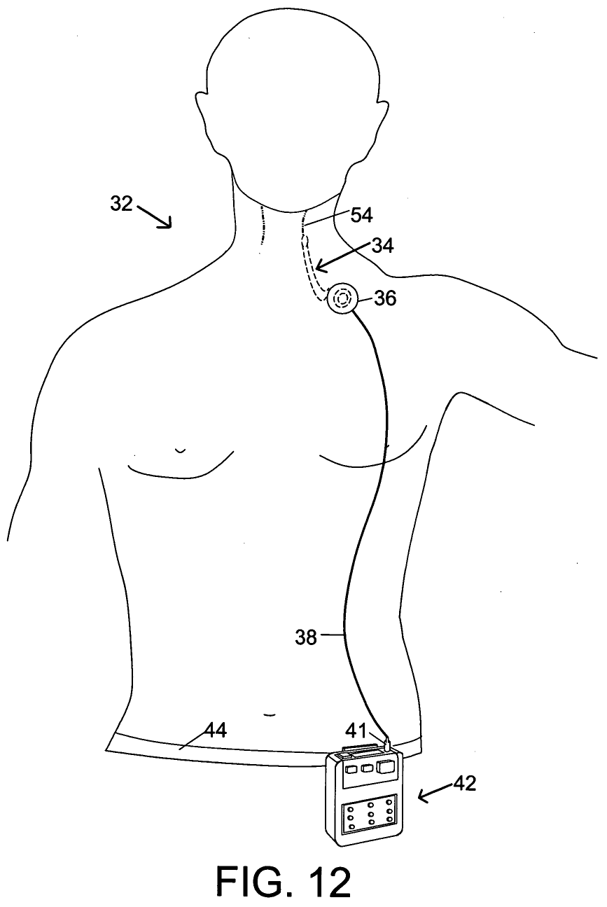

Referring to FIG. 12, which shows a schematic diagram of a patient 32 with an implantable stimulus-receiver 34 and an external stimulator 42, clipped on to a belt 44 in this case. The external stimulator 42, may alternatively be placed in a pocket or other carrying device. Shown in conjunction with FIG. 13, the primary (external) coil 46 of the external stimulator 42 is inductively coupled to the secondary (implanted) coil 48 of the implanted stimulus-receiver 34. The implantable stimulus-receiver 34 has circuitry at the proximal end 49, and has two stimulating electrodes at the distal end 61,62. The negative electrode (cathode) 61 is positioned towards the brain and the positive electrode (anode) 62 is positioned away from the brain.

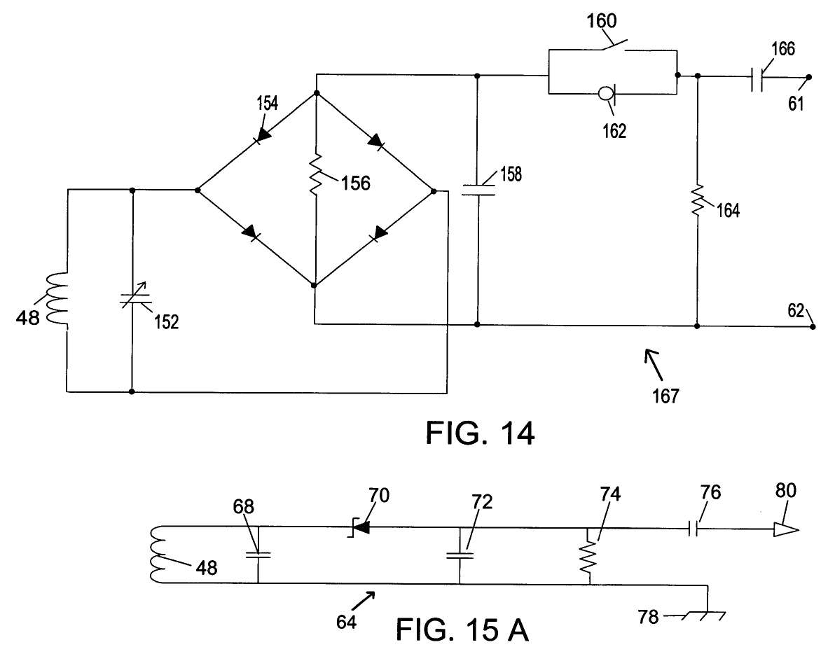

The circuitry contained in the proximal end 49 of the implantable stimulus-receiver 34 is shown schematically in FIG. 14, for one embodiment. In this embodiment, the circuit uses all passive components. Approximately 25 turn copper wire of 30 gauge, or comparable thickness, is used for the primary coil 46 and secondary coil 48. This wire is concentrically wound with the windings all in one plane. The frequency of the pulse-waveform delivered to the implanted coil 48 can vary, and so a variable capacitor 152 provides ability to tune secondary implanted circuit 167 to the signal from the primary coil 46. The pulse signal from secondary (implanted) coil 48 is rectified by the diode bridge 154 and frequency reduction obtained by capacitor 158 and resistor 164. The last component in line is capacitor 166, used for isolating the output signal from the electrode wire. The return path of signal from cathode 61 will be through anode 62 placed in proximity to the cathode 61 for “Bipolar” stimulation. In this embodiment bipolar mode of stimulation is used, however, the return path can be connected to the remote ground connection (case) of implantable circuit 167, providing for much larger intermediate tissue for “Unipolar” stimulation. Th “Bipolar” stimulation offers localized stimulation of tissue compared to “Unipolar” stimulation and is therefore, preferred in this embodiment. Unipolar stimulation is more likely to stimulate skeletal muscle in addition to nerve stimulation. The implanted circuit 167 in this embodiment is passive, so a battery does not have to be implanted.

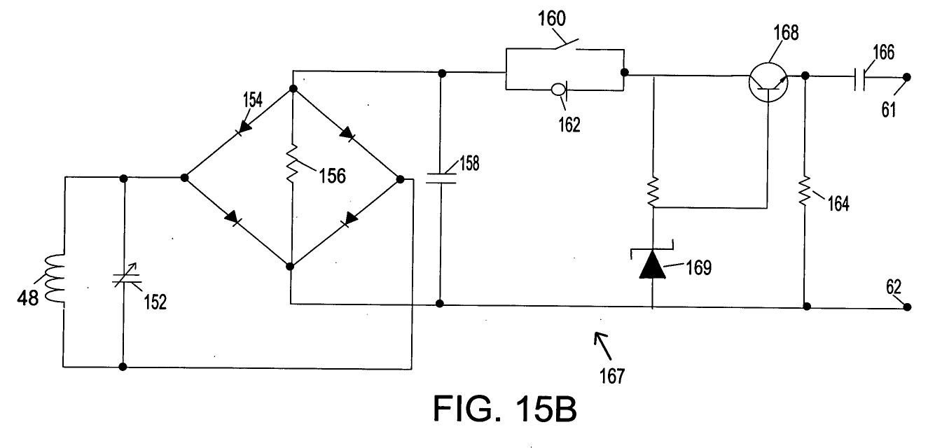

The circuitry shown in FIGS. 15A and 15B can be used as an alternative, for the implanted stimulus-receiver. The circuitry of FIG. 15A is a slightly simpler version, and circuitry of FIG. 15B contains a conventional NPN transistor 168 connected in an emitter-follower configuration.

For therapy to commence, the primary (external) coil 46 is placed on the skin 60 on top of the surgically implanted (secondary) coil 48. An adhesive tape is then placed on the skin 60 and external coil 46 such that the external coil 46, is taped to the skin 60. For efficient energy transfer to occur, it is important that the primary (external) and secondary (internal) coils 46,48 be positioned along the same axis and be optimally positioned relative to each other. In this embodiment, the external coil 46 may be connected to proximity sensing circuitry 50. The correct positioning of the external coil 46 with respect to the internal coil 48 is indicated by turning “on” of a light emitting diode (LED) on the external stimulator 42.

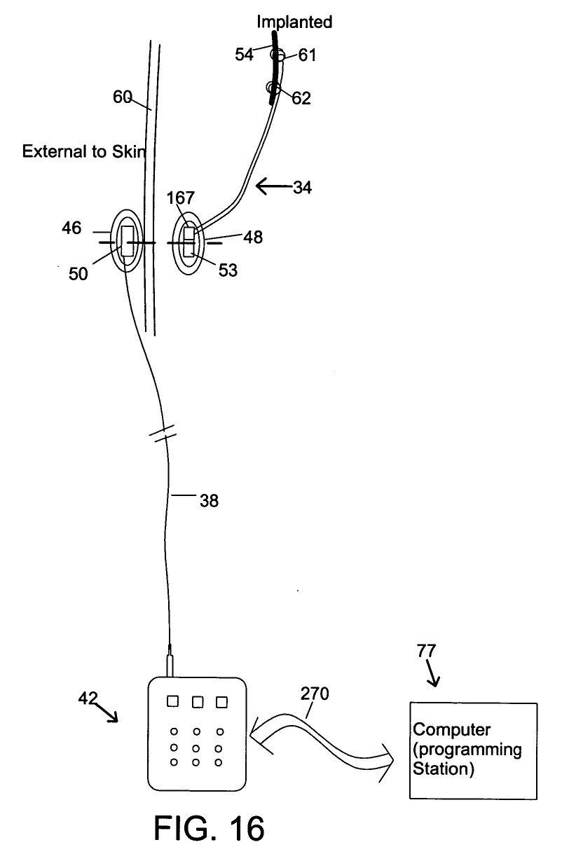

Optimal placement of the external (primary) coil 46 is done with the aid of proximity sensing circuitry incorporated in the system, in this embodiment. Proximity sensing occurs utilizing a combination of external and implantable components. The implanted components contains a relatively small magnet composed of materials that exhibit Giant Magneto-Resistor (GMR) characteristics such as Samarium-cobalt, a coil, and passive circuitry. Shown in conjunction with FIG. 16, the external coil 46 and proximity sensor circuitry 50 are rigidly connected in a convenient enclosure which is attached externally on the skin. The sensors measure the direction of the field applied from the magnet to sensors within a specific range of field strength magnitude. The dual sensors exhibit accurate sensing under relatively large separation between the sensor and the target magnet. As the external coil 46 placement is “fine tuned”, the condition where the external (primary) coil 46 comes in optimal position, i.e. is located adjacent and parallel to the subcutaneous (secondary) coil 48, along its axis, is recorded and indicated by a light emitting diode (LED) on the external stimulator 42.

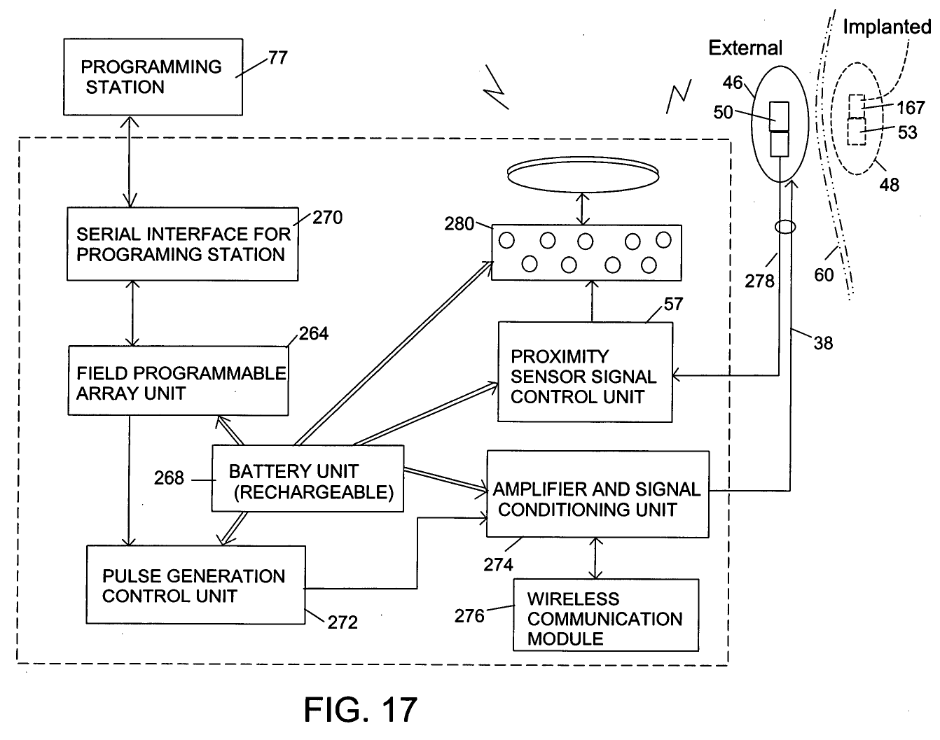

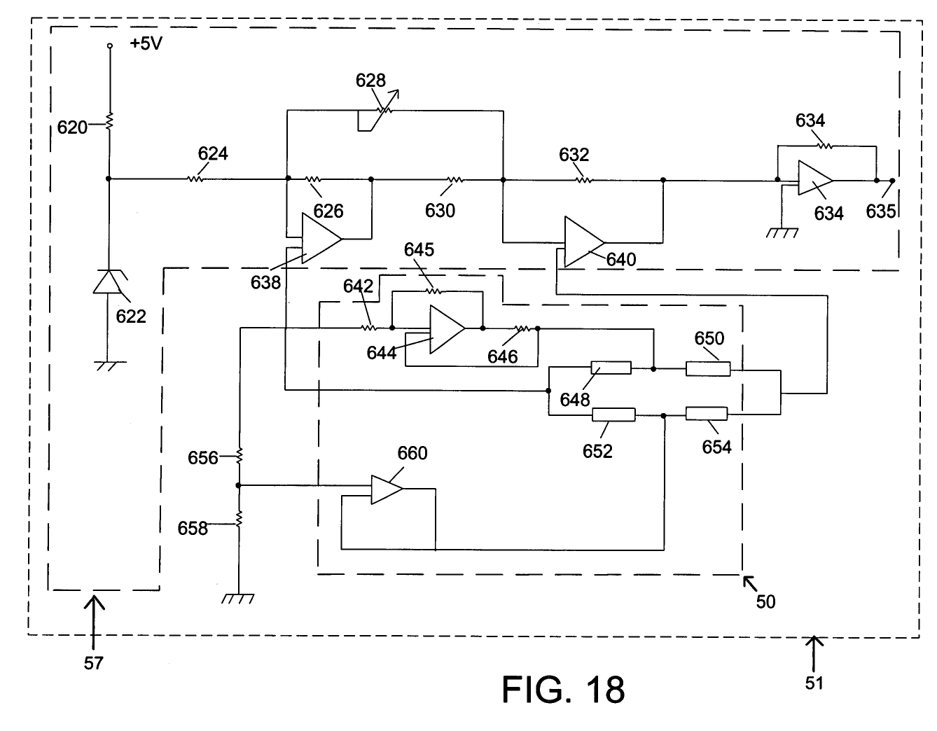

FIG. 17 shows an overall block diagram of the components of the external stimulator and the proximity sensing mechanism. The proximity sensing components are the primary (external) coil 46, supercutaneous (external) proximity sensors 648, 652 (FIG. 18) in the proximity sensor circuit unit 50, and a subcutaneous secondary coil 48 with a Giant Magneto Resister (GMR) magnet 53 associated with the proximity sensor unit. The proximity sensor circuit 50 provides a measure of the position of the secondary implanted coil 48. The signal output from proximity sensor circuit 50 is derived from the relative location of the primary and secondary coils 46, 48. The sub-assemblies consist of the coil and the associated electronic components, that are rigidly connected to the coil.

The proximity sensors (external) contained in the proximity sensor circuit 50 detect the presence of a GMR magnet 53, composed of Samarium Cobalt, that is rigidly attached to the implanted secondary coil 48. The proximity sensors, are mounted externally as a rigid assembly and sense the actual separation between the coils, also known as the proximity distance. In the event that the distance exceeds the system limit, the signal drops off and an alarm sounds to indicate failure of the production of adequate signal in the secondary implanted circuit 167, as applied in this embodiment of the device. This signal is provided to the location indicator LED 280.

FIG. 18 shows the circuit used to drive the proximity sensors 648, 652 of the proximity sensor circuit 50. The two proximity sensors 648, 652 obtain a proximity signal based on their position with respect to the implanted GMR magnet 53. This circuit also provides temperature compensation. The sensors 648, 652 are ‘Giant Magneto Resistor’ (GMR) type sensors packaged as proximity sensor unit 50. There are two components of the complete proximity sensor circuit. One component is mounted supercutaneously 50, and the other component, the proximity sensor signal control unit 57 is within the external stimulator 42. The resistance effect depends on the combination of the soft magnetic layer of magnet 53, where the change of direction of magnetization from external source can be large, and the hard magnetic layer, where the direction of magnetization remains unchanged. The resistance of this sensor 50 varies along a straight motion through the curvature of the magnetic field. A bridge differential voltage is suitably amplified and used as the proximity signal.

The Siemens GMR B6 (Siemens Corp., Special Components Inc., New Jersey) is used for this function in one embodiment. The maximum value of the peak-to-peak signal is observed as the external magnetic field becomes strong enough, at which point the resistance increases, resulting in the increase of the field-angle between the soft magnetic and hard magnetic material. The bridge voltage also increases. In this application, the two sensors 648, 652 are oriented orthogonal to each other.

The distance between the magnet 53 and sensor 50 is not relevant as long as the magnetic field is between 5 and 15 KA/m, and provides a range of distances between the sensors 648, 652 and the magnetic material 53. The GMR sensor registers the direction of the external magnetic field. A typical magnet to induce permanent magnetic field is approximately 15 by 8 by 5 mm³, for this application and these components. The sensors 648, 652 are sensitive to temperature, such that the corresponding resistance drops as temperature increases. This effect is quite minimal until about 100° C. A full bridge circuit is used for temperature compensation, as shown in temperature compensation circuit 50 of FIG. 18. The sensors 648, 652 and a pair of resistors 650, 654 are shown as part of the bridge network for temperature compensation. It is also possible to use a full bridge network of two additional sensors in place of the resistors 650, 654.

The signal from either proximity sensor 648, 652 is rectangular if the surface of the magnetic material is normal to the sensor and is radial to the axis of a circular GMR device. This indicates a shearing motion between the sensor and the magnetic device. When the sensor is parallel to the vertical axis of this device, there is a fall off of the relatively constant signal at about 25 mm. separation. The GMR sensor combination varies its resistance according to the direction of the external magnetic field, thereby providing an absolute angle sensor. The position of the GMR magnet can be registered at any angle from 0 to 360 degrees.

In the external stimulator 42 shown in FIG. 17, an indicator unit 280 which is provided to indicate proximity distance or coil proximity failure (for situations where the patch containing the external coil 46, has been removed, or is twisted abnormally etc.). Indication is also provided to assist in the placement of the patch. In case of general failure, a red light with audible signal is provided when the signal is not reaching the subcutaneous circuit. The indicator unit 280 also displays low battery status. The information on the low battery, normal and out of power conditions forewarns the user of the requirements of any corrective actions.

Also shown in FIG. 17, the programmable parameters are stored in a programmable logic 264. The predetermined programs stored in the external stimulator are capable of being modified through the use of a separate programming station 77. The Programmable Array Logic Unit 264 and interface unit 270 are interfaced to the programming station 77. The programming station 77 can be used to load new programs, change the existing predetermined programs or the program parameters for various stimulation programs. The programming station is connected to the programmable array unit 75 (comprising programmable array logic 304 and interface unit 270) with an RS232-C serial connection. The main purpose of the serial line interface is to provide an RS232-C standard interface.

This method enables any portable computer with a serial interface to communicate and program the parameters for storing the various programs. The serial communication interface receives the serial data, buffers this data and converts it to a 16 bit parallel data. The programmable array logic 264 component of programmable array unit receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit 264, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the pre-determined programs is well known to those skilled in the art.

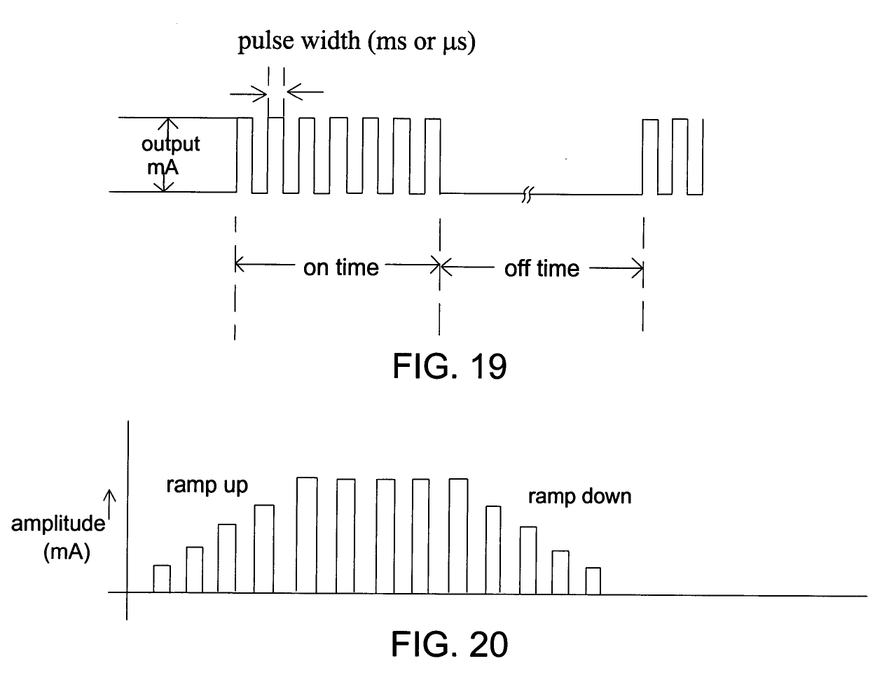

The pulses delivered to the nerve tissue for stimulation therapy are shown graphically in FIG. 19. As shown in FIG. 20, for patient comfort when the electrical stimulation is turned on, the electrical stimulation is ramped up and ramped down, instead of abrupt delivery of electrical pulses.

The selective stimulation to the vagus nerve can be performed in one of two ways. One method is to activate one of several “pre-determined” programs. A second method is to “custom” program the electrical parameters which can be selectively programmed, for specific therapy to the individual patient. The electrical parameters which can be individually programmed, include variables such as pulse amplitude, pulse width, frequency of stimulation, stimulation on-time, and stimulation off-time. Table two below defines the approximate range of parameters,

TABLE 2
Electrical parameter range delivered to the nerve

| PARAMER | RANGE |
|---|---|
| Pulse Amplitude | 0.1 Volt-10 Volts |
| Pulse width | 20 μS-5 mSec. |
| Frequency | 5 Hz-200 Hz |
| On-time | 10 Secs-24 hours |
| Off-time | 10 Secs-24 hours |

The parameters in Table 2 are the electrical signals delivered to the nerve via the two electrodes 61,62 (distal and proximal) around the nerve, as shown in FIG. 13. It being understood that the signals generated by the external pulse generator 42 and transmitted via the primary coil 46 are larger, because the attenuation factor between the primary coil and secondary coil is approximately 10-20 times, depending upon the distance, and orientation between the two coils. Accordingly, the range of transmitted signals of the external pulse generator are approximately 10-20 times larger than shown in Table 2.

Referring now to FIG. 21, the implanted lead component of the system is similar to cardiac pacemaker leads, except for distal portion (or electrode end) of the lead. The lead terminal preferably is linear bipolar, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes 61,62 for stimulating the vagus nerve 54 may either wrap around the nerve once or may be spiral shaped. These stimulating electrodes may be made of pure platinum, platinum/Iridium alloy or platinum/iridium coated with titanium nitride. The conductor connecting the terminal to the electrodes 61,62 is made of an alloy of nickel-cobalt. The implanted lead design variables are also summarized in table three below.

TABLE 3
Lead design variables

| | |
|---|---|
| Proximal | Distal |
| End | End |

| | | | Conductor | | |
|---|---|---|---|---|---|
| | | | (connecting | | |
| | Lead body- | | proximal | | |
| Lead | Insulation | | and distal | Electrode - | Electrode - |
| Terminal | Materials | Lead-Coating | ends) | Material | Type |
| Linear | Polyurethane | Antimicrobial | Alloy of | Pure | Spiral |
| bipolar | | coating | Nickel- | Platinum | electrode |
| | | | Cobalt | | |
| Bifurcated | Silicone | Anti- | | Platinum- | Wrap-around |
| | | Inflamatory | | Iridium | electrode |
| | | coating | | (Pt/Ir) Alloy | |
| | Silicone with | Lubricious | | Pt/Ir coated | Steroid |
| | Polytetrafluoroethylene | coating | | with Titanium | eluting |
| | (PTFE) | | | Nitride | |
| | | | | Carbon | |

Once the lead is fabricated, coating such as anti-microbial, anti-inflammatory, or lubricious coating may be applied to the body of the lead.

### Implanted Stimulus-Receiver Comprising a High Value Capacitor for Storing Charge Used in Conjunction With an External Stimulator

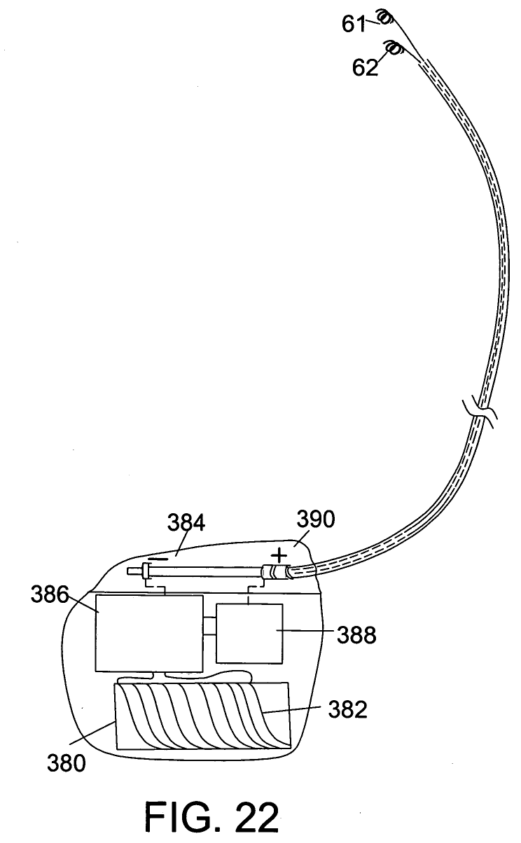

In one embodiment, the implanted stimulus-receiver may be a system which is RF coupled combined with a power source. In this embodiment, the implanted stimulus-receiver contains high value, small sized capacitor(s) for storing charge and delivering electric stimulation pulses for up to several hours by itself, once the capacitors are charged. The packaging is shown in FIG. 22. Using mostly hybrid components and appropriate packaging, the implanted portion of the system described below is conducive to miniaturization. As shown in FIG. 22, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment.

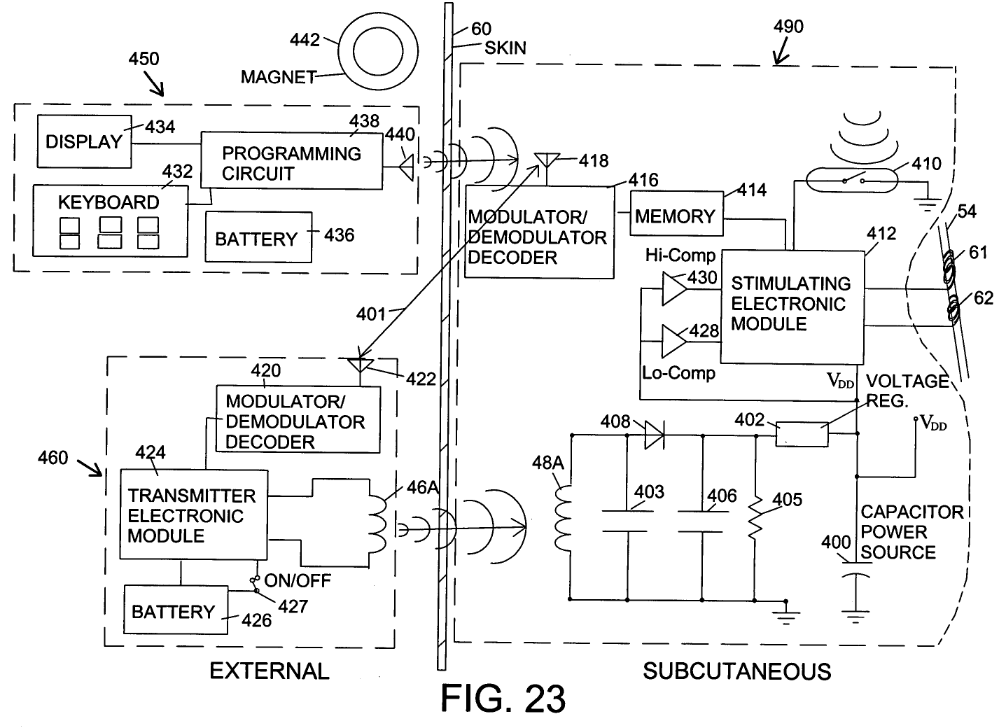

As shown in conjunction with FIG. 23 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641.

The refresh-recharge transmitter unit 460 includes a primary battery 426, an ON/Off switch 427, a transmitter electronic module 442, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 442 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is V_{DD} reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lₘ which pulls reed switch 410 closed. Upon closure of reed switch 410, stimulating electronic module 412 in conjunction with memory 414 begins the delivery (or cessation as the case may be) of controlled electronic stimulation pulses to the vagus nerve 54 via electrodes 61, 62. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

### Programmer-Less Implantable Pulse Generator (IPG)

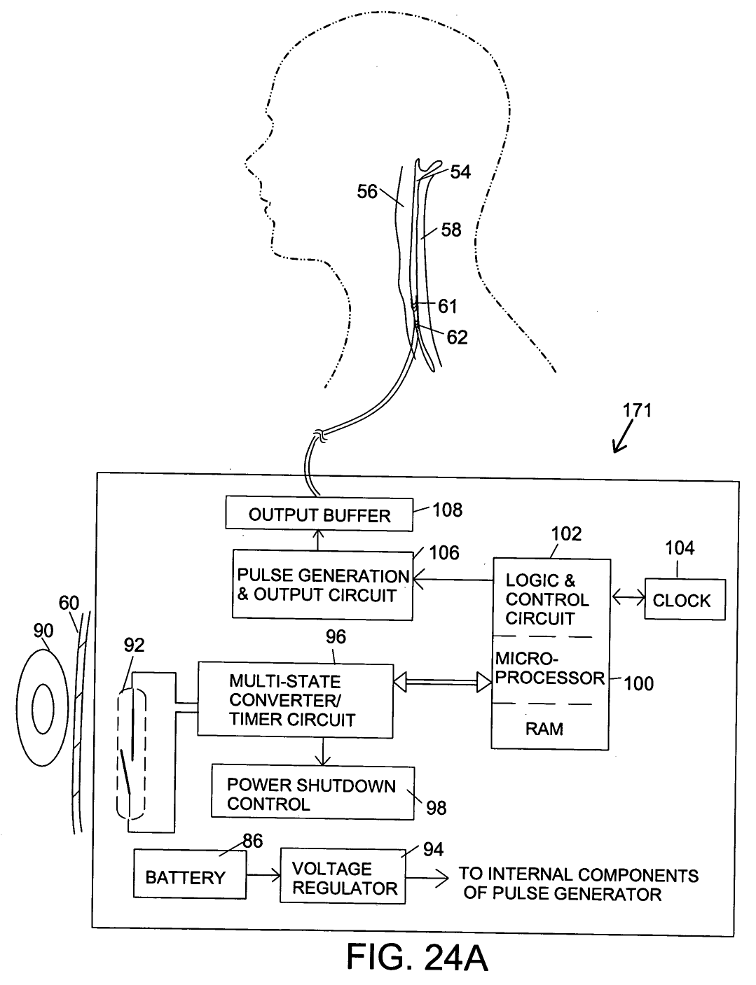

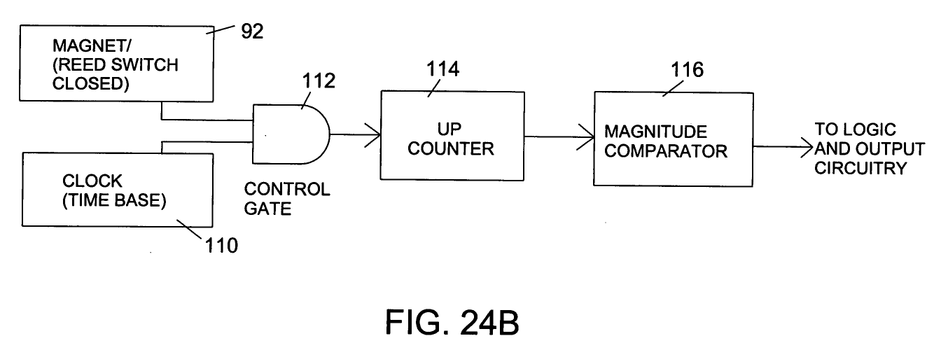

In one embodiment, a programmer-less implantable pulse generator (IPG) may be used. In this embodiment, shown in conjunction with FIG. 24A, the implantable pulse generator 171 is provided with a reed switch 92 and memory circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

In one embodiment, shown in conjunction with FIG. 24B, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, as shown in FIG. 24A, the pulse generation and amplification circuit 106 deliver the appropriate electrical pulses to the vagus nerve 54 of the patient via an output buffer 108. The delivery of output pulses is configured such that the distal electrode 61 (electrode closer to the brain) is the cathode and the proximal electrode 62 is the anode. Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor may be a commercially available, general purpose microprocessor or microcontroller, or may be a custom integrated circuit device augmented by standard RAM/ROM components.

In one embodiment, there are four stimulation states. A larger (or lower) number of states can be achieved using the same methodology, and such is considered within the scope of the invention. These four states are, LOW stimulation state, LOW-MED stimulation state, MED stimulation state, and HIGH stimulation state. Examples of stimulation parameters (delivered to the vagus nerve) for each state are as follows,

LOW stimulation state example is,

| | |
|---|---|
| Current output: | 0.75 milliAmps. |
| Pulse width: | 0.20 msec. |
| Pulse frequency: | 20 Hz |
| Cycles: | 20 sec. on-time and 2.0 min. off-time in |
| | repeating cycles. |

LOW-MED stimulation state example is,

| | |
|---|---|
| Current output: | 1.5 milliAmps, |
| Pulse width: | 0.30 msec. |
| Pulse frequency: | 25 Hz |
| Cycles: | 1.5 min. on-time and 20.0 min. off-time in |
| | repeating cycles. |

MED stimulation state example is,

| | |
|---|---|
| Current output: | 2.0 milliAmps. |
| Pulse width: | 0.30 msec. |
| Pulse frequency: | 30 Hz |
| Cycles: | 1.5 min. on-time and 20.0 min. off-time in |
| | repeating cycles. |

HIGH stimulation state example is,

| | |
|---|---|
| Current output: | 3.0 milliAmps, |
| Pulse width: | 0.40 msec. |
| Pulse frequency: | 30 Hz |
| Cycles: | 2.0 min. on-time and 20.0 min. off-time in |
| | repeating cycles. |

These prepackaged/predetermined programs are mearly examples, and the actual stimulation parameters will deviate from these depending on the treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a prepackaged/predetermined program such as presented above. In such an embodiment, the system could be configured such that one application of the magnet triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, triggers the device OFF.

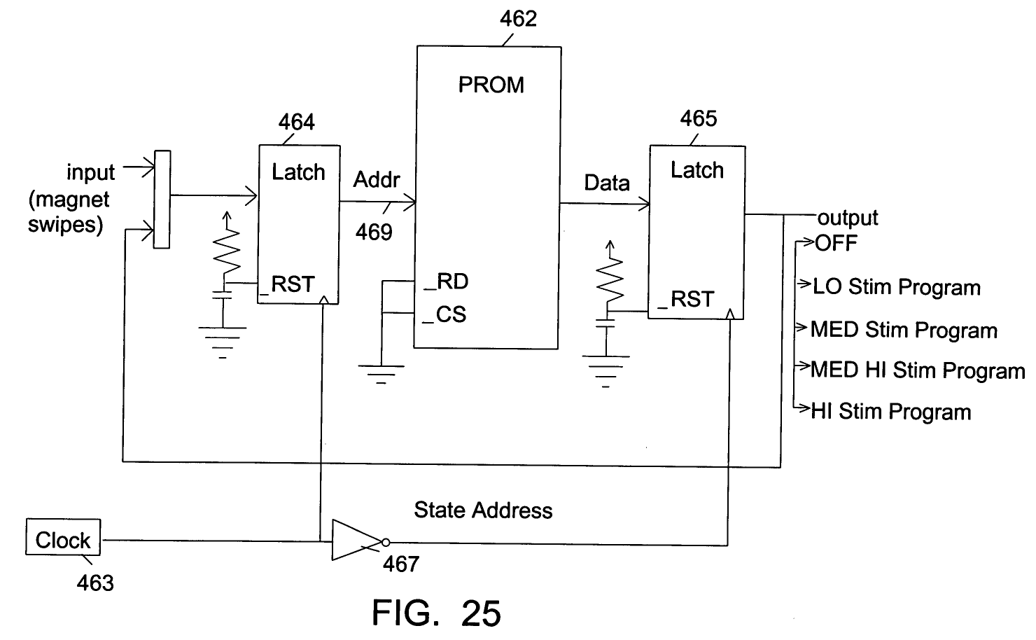

FIG. 25 shows a representative digital circuitry used for the basic state machine circuit. The circuit consists of a PROM 462 that has part of its data fed back as a state address. Other address lines 469 are used as circuit inputs, and the state machine changes its state address on the basis of these inputs. The clock 463 is used to pass the new address to the PROM 462 and then pass the output from the PROM 462 to the outputs and input state circuits. The two latches 464, 465 are operated 180° out of phase to prevent glitches from unexpectedly affecting any output circuits when the ROM changes state. Each state responds differently according to the inputs it receives.

The advantage of this embodiment is that it is cheaper to manufacture than a fully programmable implantable pulse generator (IPG).

### Programmable Implantable Pulse Generator (IPG)

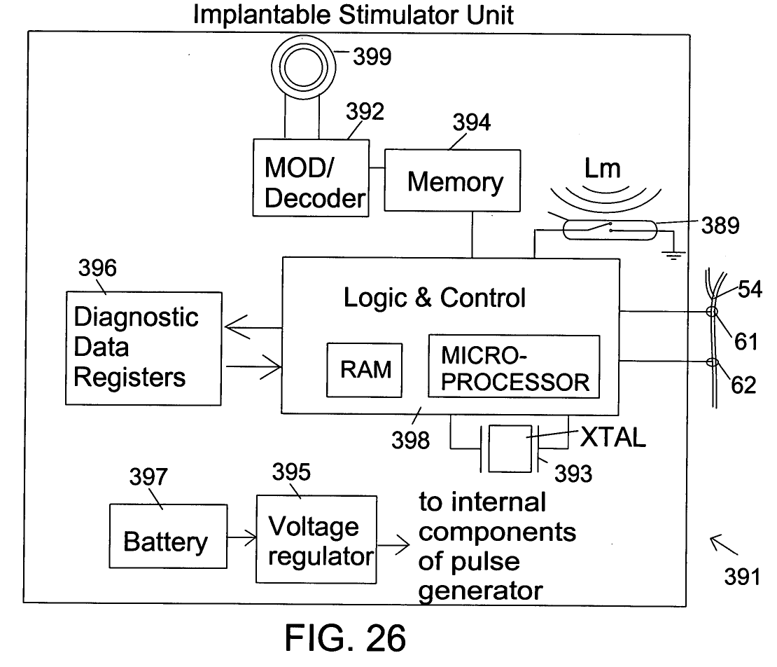

In one embodiment, a fully programmable implantable pulse generator (IPG) may be used. Shown in conjunction with FIG. 26, the implantable stimulator unit 391 is preferably a microprocessor based device, where the entire circuitry is encased in a hermetically sealed titanium can. Once programmed via an external programmer, the implanted pulse generator 391 provides appropriate electrical stimulation pulses to the vagus nerve 54 via electrodes 61,62. The range of programmable electrical stimulation parameters are shown in table 4 below.

TABLE 4
Programmable electrical parameter range

| PARAMER | RANGE |
|---|---|
| Pulse Amplitude | 0.1 Volt-10 Volts |
| Pulse width | 20 μS-5 mSec. |
| Frequency | 3 Hz-300 Hz |
| On-time | 5 Secs-24 hours |
| Off-time | 5 Secs-24 hours |
| Ramp | ON/OFF |

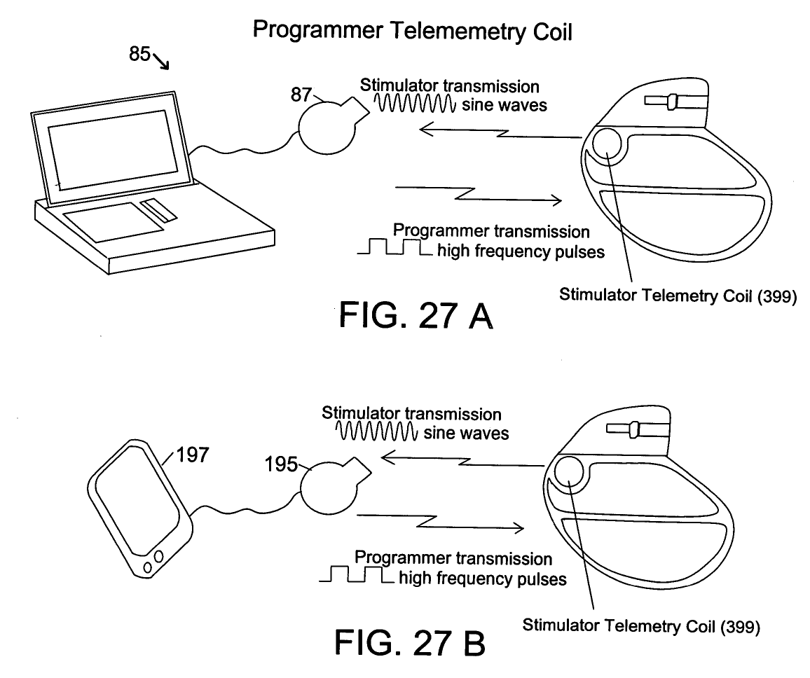

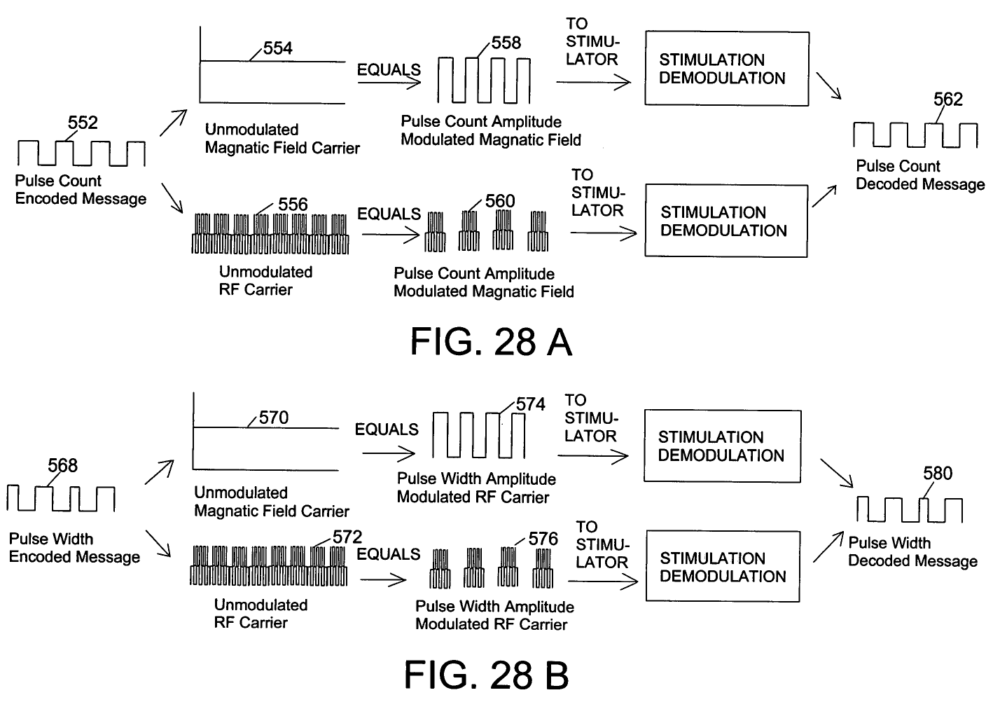

Device interrogation and programming pulses are provided via a telemetry coil 399. Programming pulses are decoded by a decoder 392 and stored in the memory 394 of the pulse generator 391. In this embodiment, an implanted battery 397 supplies power to all internal components of the pulse generator 391. The programming of the IPG 391 is shown in conjunction with FIGS. 27A and 27B. With the magnetic Reed Switch 389 in the closed position, a coil in the head of the programmer, communicates with a telemetry coil 399 (shown in FIG. 26) of the implanted pulse generator 391. Bi-directional inductive telemetry is used to exchange data with the implanted unit 391 by means of the external programming unit 85. Shown in conjunction with FIGS. 27A, 27B, 28A and 28B, inductive coupling is employed to transmit the programming instructions, which are detected by the antenna coil 399. These pulses of the magnetic field ar transmitted in a coding scheme that induces current to flow in the antenna coil 399. Programming takes place via coil 399, a receiving amplifier, a decoder, a controller, and the register in which the temporary and permanent programs are stored. Radiofrequency (RF) waves of electromagnetic field using frequencies of approximately 100 KHz, that allow rapid transmission of large amounts of information, The RF frequency is modulated, allowing the encoding of information during transmission by the programmer 85. The receiver coil 399 is tuned through properly selected inductor-capacitor values to have unique sensitivity to the carrier frequency of the transmitted signals.

The transmission of programming information involves manipulation of the carrier signal in a manner that is recognizable by the pulse generator 391 as a valid set of instructions. The process of modulation serves as a means of encoding the programming instruction in a language that is interpretable by the pulse generator. Modulation of signal amplitude, pulse width, and time between pulses are all used in the programming system, as will be appreciated by those skilled in the art. FIG. 28A shows an example of pulse count modulation, and FIG. 28B shows an example of pulse width modulation.

### Combination Implantable Device Comprising Both a Stimulus-Receiver and a Programmable Implantable Pulse Generator (IPG)

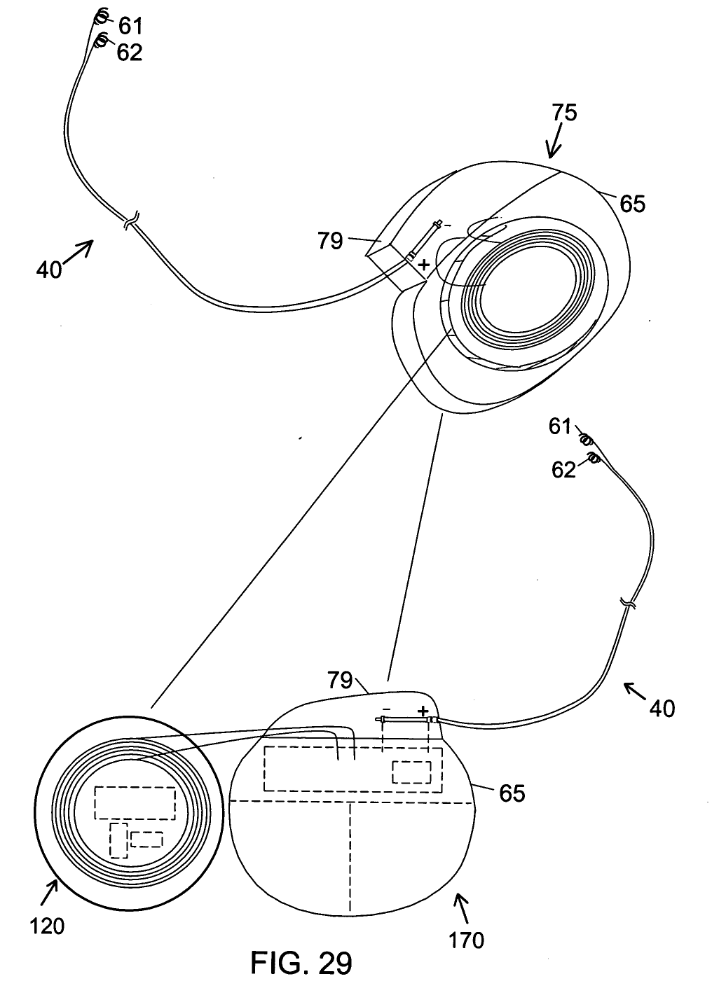

In one embodiment, the implantable device may comprise both a stimulus-receiver and a programmable implantable pulse generator (IPG). FIG. 29 shows a close up view of the packaging of the implanted stimulator 75 of this embodiment, showing the two subassemblies 120, 170. The two subassemblies are the stimulus-receiver module 120 and the battery operated pulse generator module 170. The external stimulator 42, and programmer 85 also being remotely controllable from a distant location via the internet. Controlling circuitry means within the stimulator 75, makes the inductively coupled stimulator 120 and the IPG 170 operate in harmony with each other. For example, when stimulation is applied via the inductively coupled system, the battery operated portion of the stimulator is triggered to go into the “sleep” mode. Conversely, when programming pulses (which are also inductively coupled) are being applied to the implanted battery operated pulse generator 170, the inductively coupled stimulation circuitry 120 is disconnected.

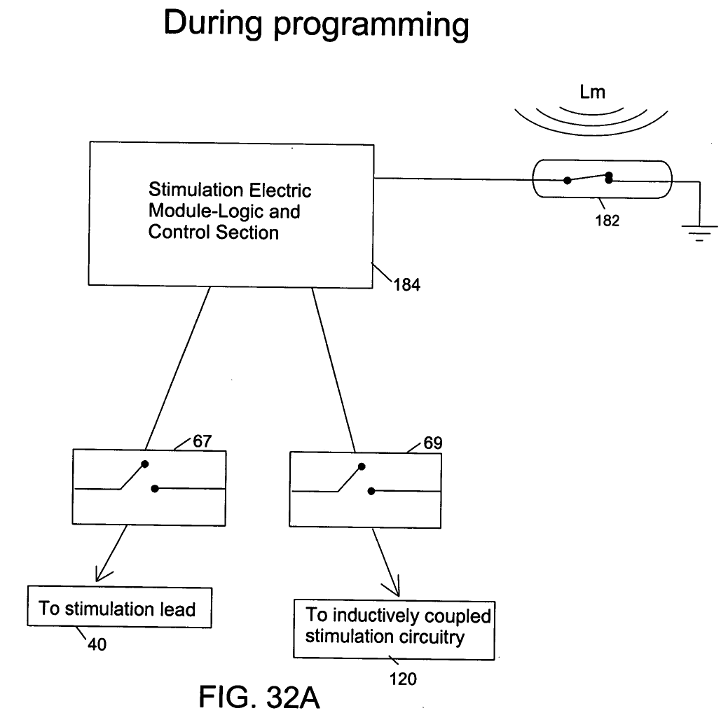

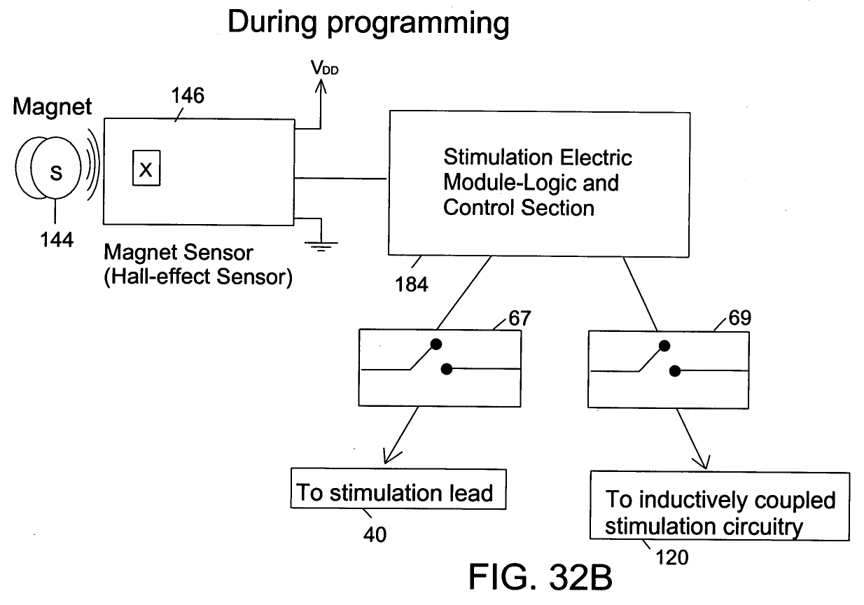

FIG. 32A is a simplified diagram of one aspect of control circuitry. In this embodiment, to program the implanted portion of the stimulator 70, a magnet 144 is placed over the implanted pulse generator 170, causing a magnetically controlled Reed Switch 182 (which is normally in the open position) to be closed. As is also shown in FIG. 32A, at the same time a switch 67 going to the stimulator lead 40, and a switch 69 going to the circuit of the stimulus-receiver module 120 are both opened, completely disconnecting both subassemblies electrically. Further, protection circuitry 181 is an additional safeguard for inadvertent leakage of electrical energy into the nerve tissue 54 during programming. Alternatively, as shown in FIG. 32B, instead of a reed switch 182, a solid state magnet sensor (Hall-effect sensor) 146 may be used for the same purpose. In one embodiment, the solid state magnet sensor 146 is preferred, since there are no moving parts that can get stuck.

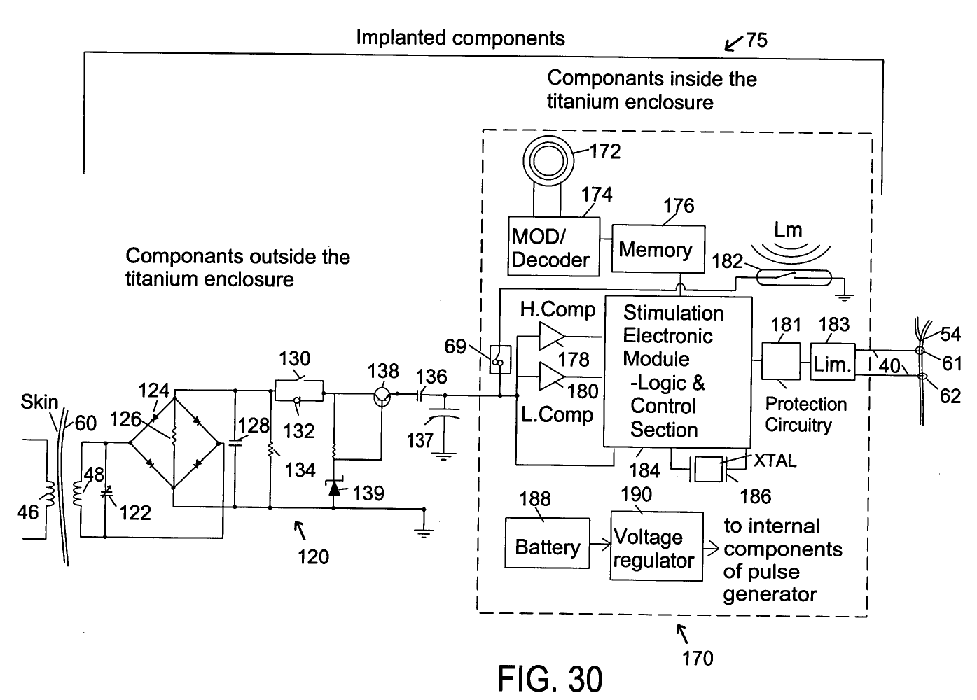

With reference to FIG. 30, for the functioning of the inductively coupled stimulus-receiver 120, a primary (external) coil 46 is placed in close proximity to secondary (implanted) coil 48. The primary coil 46 may be taped to skin 60, or other means may be used for keeping the primary coil in close proximity. Referring to the left portion of FIG. 30, the amplitude and pulse width modulated radiofrequency signals from the primary (external) coil 46 are electromagnetically coupled to the secondary (implanted) coil 48 in the implanted unit 75. The two coils 46 and 48 thus act like an air-gap transformer. The system having means for proximity sensing between the two coils 46,48, and feedback regulation of signals as described earlier.

Again with reference to FIG. 30, the combination of capacitor 122 and inductor 48 tunes the receiver circuitry to the high frequency of the transmitter with the capacitor 122. The receiver is made sensitive to frequencies near the resonant frequency of the tuned circuit and less sensitive to frequencies away from the resonant frequency. A diode bridge 124 rectifies the alternating voltages. Capacitor 128 and resistor 134 filter out the high-frequency component of the receiver signal, and leaves the current pulse of the same duration as the bursts of the high-frequency signal. A zenor diode 139 is used for regulation and capacitor 136 blocks any net direct current.

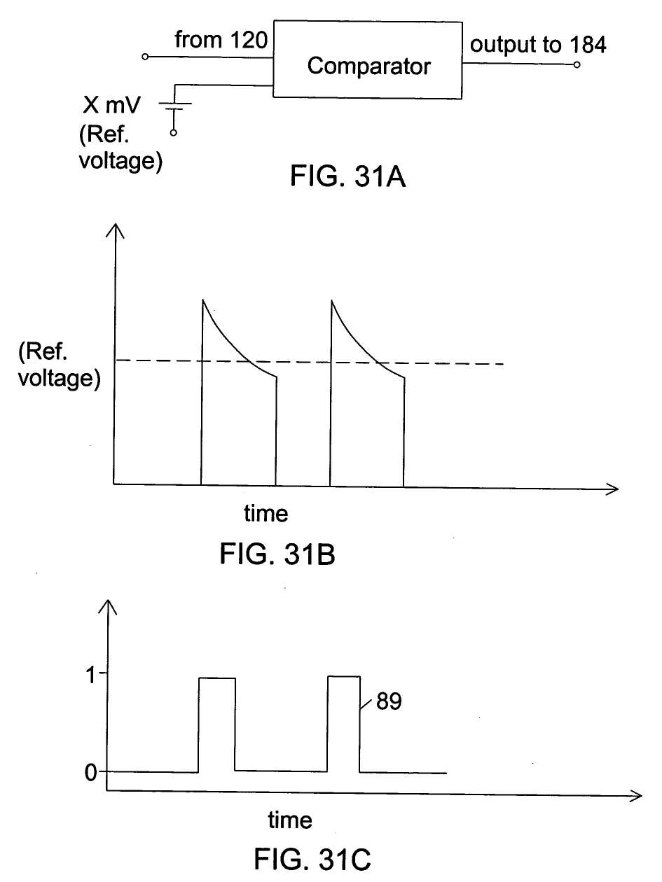

As shown in conjunction with FIGS. 30 and 31 the pulses generated from the stimulus-receiver circuitry 120 ar compared to a reference voltage, which is programmed in the implanted pulse generator 170. When the voltage of incoming pulses exceeds the reference voltage (FIG. 31 B), the output of the comparator 178,180 sends digital pulse 89 (shown in FIG. 31C) to the stimulation electric module 184. At this predetermined level, the high threshold comparator 178 fires and the controller 184 suspends any stimulation from the implanted pulse generator 170. The implanted pulse generator 170 goes into “sleep” mode for a predetermined period of time. In one preferred embodiment, the level of voltage needed for the battery operated stimulator to go into “sleep” mode is a programmable parameter. The length of time, the implanted pulse generator 170 remains in “sleep” mode is also a programmable parameter. Therefore, advantageously the external stimulator 42 in conjunction with the inductively coupled part of the stimulator 120 can be used to save the battery life of the implanted stimulator 75.

In one embodiment, the external stimulator 42 is networked using the internet, giving the attending physician full control for activating and de-activating selected programs. Using “trial and error” various programs for electrical pulse therapy can be custom adjusted for the physiology of the individual patent. Also, by using the external stimulator 42, the battery 188 of the implanted stimulator unit 75 can be greatly extended. Further, even after the battery 188 is depleted, the system can still be used for neuromodulation using the stimulus-receiver module 120, and the external stimulator 42.

At some point, the implanted pulse generator 170 is programmed with the external programmer 85, using a modified PC and a programming wand 87, as is shown in FIGS. 27A and 27B.

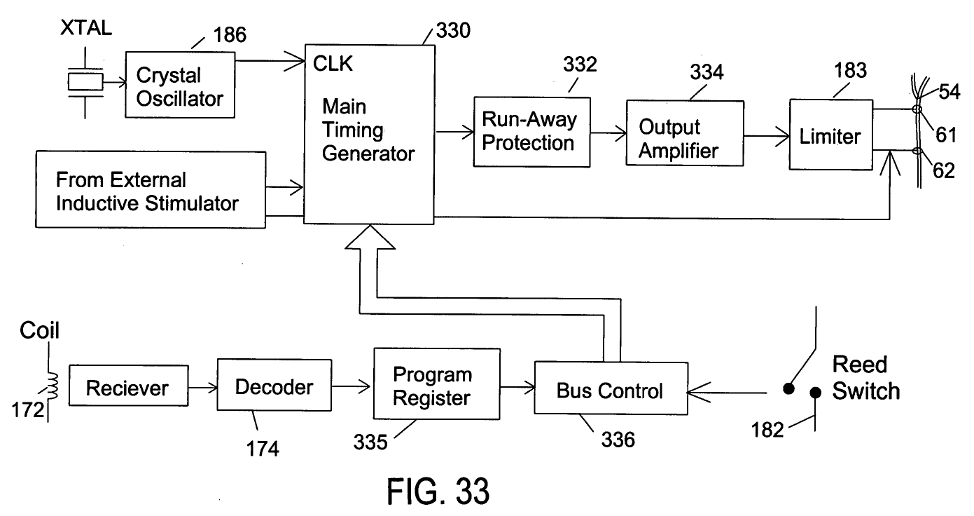

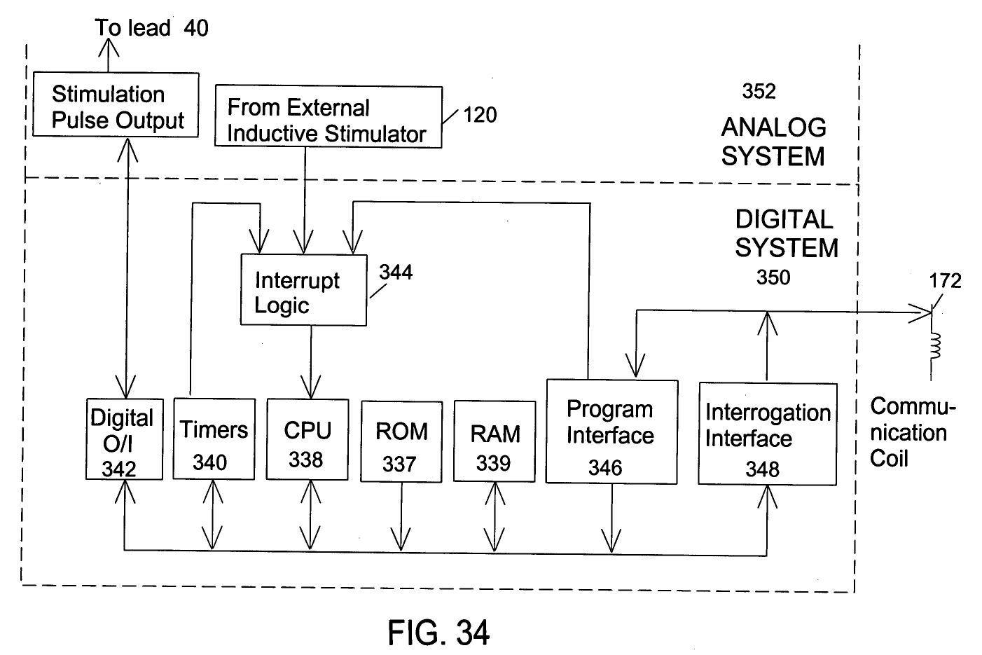

The battery-operated portion of the system 170 is shown on the right side of FIG. 30, and is described in conjunction with FIGS. 33 and 34. The stimulation electronic module 184 comprises both digital and analog circuits. The main timing generator 330 (shown in FIG. 33), controls the timing of the analog output circuitry for delivering neuromodulating pulses to the vagus nerve 54, via output amplifier 334. Limiter 183 prevents excessive stimulation energy from getting into the vagus nerve 54. The main timing generator 330 receiving clock pulses from crystal oscillator 186. Main timing generator 330 also receiving input from inductively coupled circuitry 120, and programmer 85 via coil 172. FIG. 34 highlights other portions of the digital system such as CPU 338, ROM 337, RAM 339, program interface 346, interrogation interface 348, timers 340, and digital O/I 342.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil situated under the hybrid substrate is used for bidirectional telemetry. For the implanted battery portion 170, the hybrid and battery 188 are encased in a titanium can 65. This housing is a two-part titanium capsule that is hermetically sealed by laser welding. Alternatively, electron-beam welding can also be used. The header 79 (FIG. 29) is a cast epoxy-resin with hermetically sealed feedthrough, and form the lead 40 connection block. The stimulus-receiver assembly 120 is then also assembled on to the pulse generator 170 to finish the complete implanted stimulator 75.

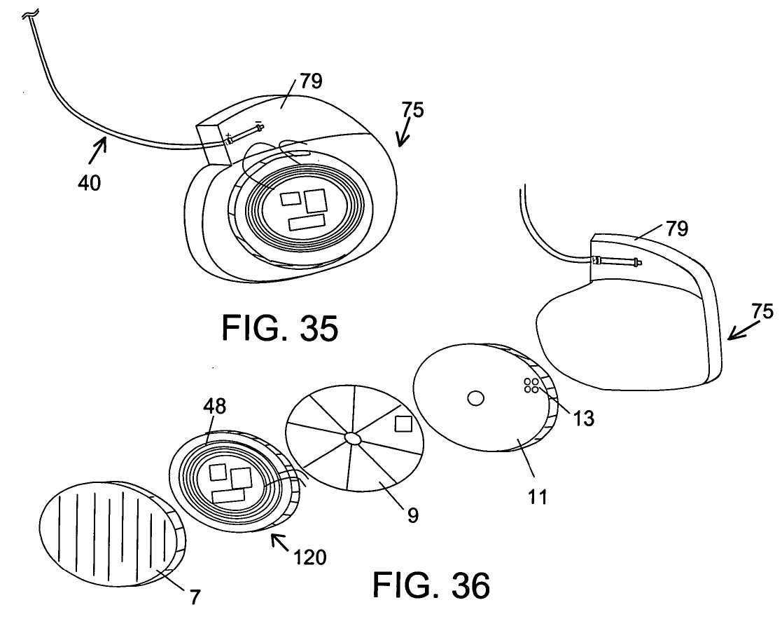

FIG. 35 shows a diagram of the finished implantable stimulator 75. FIG. 36 shows the pulse generator with some of the components used in assembly in an exploded view. These components include a coil cover 7, the secondary coil 48 and associated components, a magnetic shield 9, and a coil assembly carrier 11. The coil assembly carrier 11 has at least one positioning detail 13 located between the coil assembly and the feed through for positioning the electrical connection. The positioning detail 13 secures the electrical connection.

### Implantable Pulse Generator (IPG) Comprising a Rechargable Battery

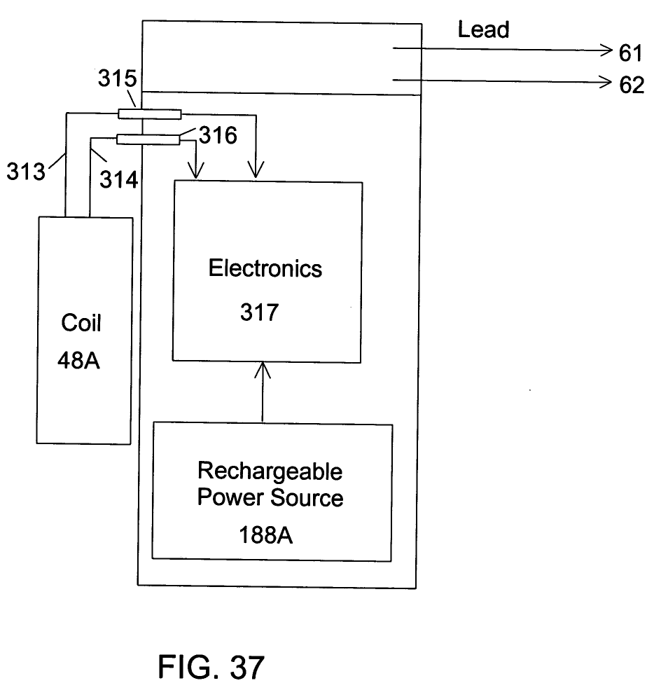

In one embodiment, an implantable pulse generator with rechargeable power source can be used. In such an embodiment (shown in conjunction with FIG. 37), a recharge coil is external to the pulse generator titanium can. The RF pulses transmitted via coil 46 and received via subcutaneous coil 48A are rectified via diode bridge 154. These DC pulses are processed and the resulting current applied to recharge the battery 188A in the implanted pulse generator.

In summery, the method of the current invention can be practiced with any of the several power sources disclosed above.

In one embodiment, the external stimulator has a telecommunications module, as described in a co-pending application, and summarized here for reader convenience. The telecommunications module has two-way communications capabilities.

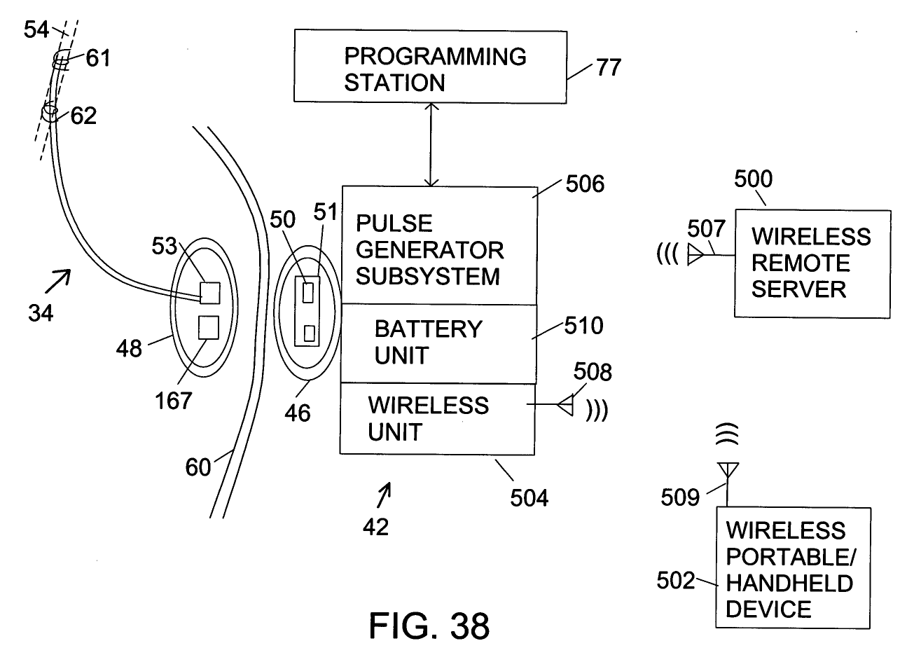

FIG. 38 shows the communication between the external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, Calif.) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

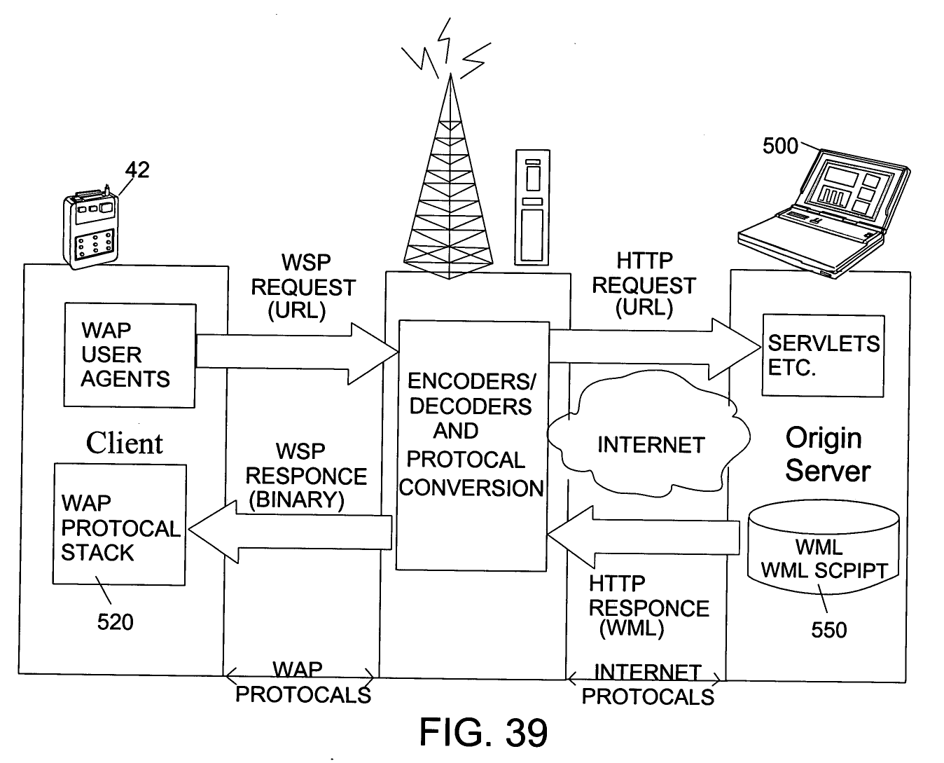

In one aspect of the invention, the telecommunications component can use Wireless Application Protocol (WAP). The Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model, and is shown schematically in FIG. 39. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

The key components of the WAP technology, as shown in FIG. 39, includes 1) Wireless Mark-up Language (WML) 550 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WMLScript content. 4) A lightweight protocol stack 520 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

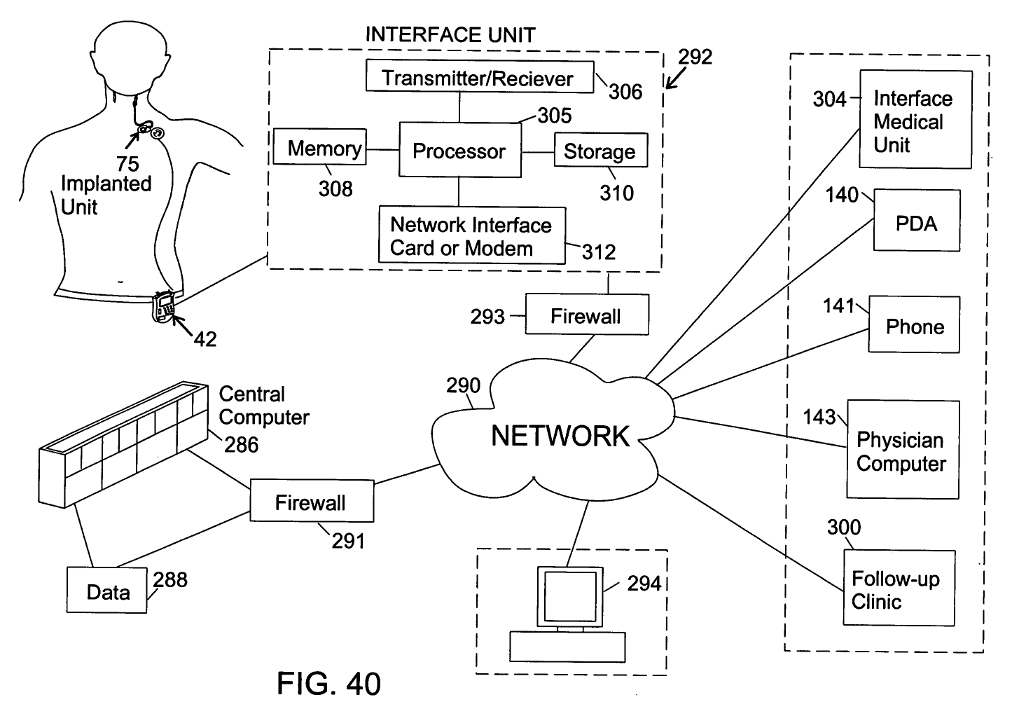

Shown in conjunction with FIG. 40, in one embodiment, the external stimulator 42 and/or the programmer (85 in FIG. 27A, or 197 in FIG. 27B) may be networked to a central collaboration computer 286 as well as other devices such as a remote computer 294, PDA 140, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

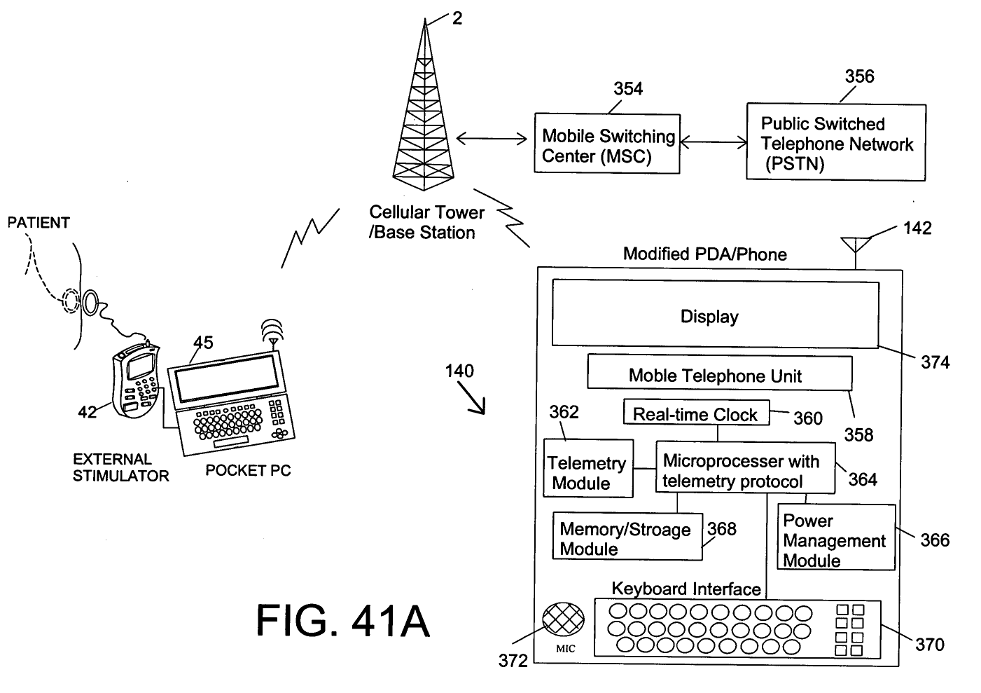

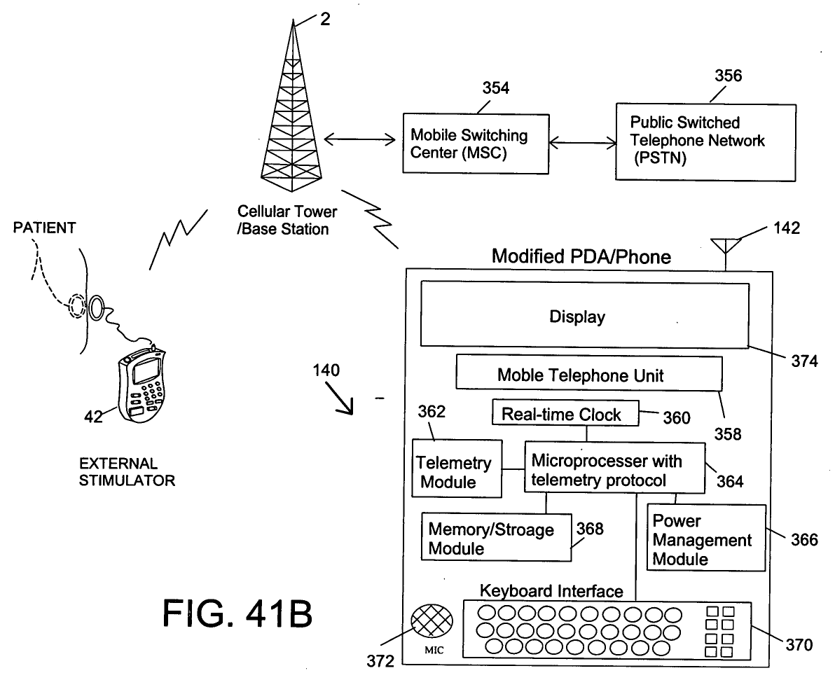

Shown in conjunction with FIGS. 41A and 41B the physician's remote communication's module is a Modified PDA/Phone 140 in this embodiment. The Modified PDA/Phone 140 is a microprocessor based device as shown in a simplified block diagram in FIGS. 41A and 41B. The PDA/Phone 140 is configured to accept PCM/CIA cards specially configured to fulfill the role of communication module 292 of the present invention. The Modified PDA/Phone 140 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 140 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 140 and external stimulator 42. The intent of this invention is to use 3 G technology for wireless communication and data exchange, even though in some cases 2.5 G is being used currently.

For the system of the current invention, the use of any of the “3 G” technologies for communication for the Modified PDA/Phone 140, is considered within the scope of the invention. Further, it will be evident to one of ordinary skill in the art, that as future 4 G systems, which will include new technologies such as improved modulation and smart antennas, can be easily incorporated into the system and method of current invention, and are also considered within the scope of the invention.

1. A method of providing electrical pulses to one or both vagus nerve(s) and its branches of a patient to provide therapy for at least one of atrial fibrillation, congestive heart failure, inappropriate sinus tachycardia, and refractory hypertension, comprising the steps of:
a) providing a stimulation means, wherein said stimulation means comprising implantable and external components;
b) providing programmer means, wherein said programmer means comprising means for networking with remote computers for data exchange; and
c) programming said stimulation means with said programming means;
whereby, said therapy is provided by said electrical pulses.

2. The method of claim 1, wherein said stimulation means comprises an implantable pulse generator with at least two fixed programs which are activated with a magnet.

3. The method of claim 1, wherein said external component is an external magnet.

4. The method of claim 1, wherein said external component is an external stimulator.

5. The method of claim 4, wherein said external stimulator further comprises telemetry means for networking.

6. The method of claim 1, wherein said programmer further comprises a telemetry unit for networking.

7. The method of claim 6, wherein said programmer means can be remotely operated over a wide area network.

8. The method of claim 1, wherein said stimulation means comprises,
a) an implanted stimulus-receiver; said stimulus-receiver comprising circuitry and a high-value capacitor for storing charge; and
b) an external stimulator for delivering power and data.

9. The method of claim 1, wherein said implantable components comprise an implantable pulse generator (IPG) with a recharging coil for recharging the implantable pulse generator using an external power source.

10. A method of providing electrical pulses to one or both vagus nerve(s) and its branches of a patient, with a stimulation means comprising implanted and external components to provide therapy for at least one of atrial fibrillation, congestive heart failure, inappropriate sinus tachycardia, and refractory hypertension, comprising the steps of:
a) providing implantable pulse generator means;
b) providing an external stimulator means and programming means;
c) providing a lead in connection with said implantable pulse generator means, and adapted to be in contact with the said vagus nerve(s); and
d) selectively operating said implantable pulse generator means or external stimulator means
whereby, said therapy is provided with pulsed electrical stimulation.

11. The method of claim 10, wherein said programmer means are remotely operated a wide area network.

12. The method of claim 10, wherein said external stimulation means are remotely controlled over a wide area network.

13. A method of providing therapy for congestive heart failure (CHF) using electrical pulses to a vagus nerve, comprising the steps of:
a) providing implantable stimulation means wherein, said stimulation means comprises implanted or external power source, to provid electrical pulses to said vagus nerve;
b) providing programmer means external to the body for programming said stimulation means;
whereby, said electrical pulses supplied to said vagus nerve provide therapy for congestive heart failure.

14. The method of claim 13, wherein said stimulation means comprises an implantable pulse generator with fixed programs which is controllable with a magnet.

15. The method of claim 13, wherein said programmer means are remotely operated over a wide area network.

16. The method of claim 13, wherein said stimulation means can be remotely controlled over a wide area network.

17. The method of claim 13, wherein said implantable components comprise an implantable pulse generator (IPG) with a recharging coil for recharging the implantable pulse generator using an external power source

18. A method to increase the cardiac parasympathetic tone in a patient using pulsed electrical stimulation to a vagus nerve, comprising the steps of:
a) providing implantable stimulation means wherein, said stimulation means comprises implanted or external power source, to provide electrical pulses to said vagus nerve;
b) providing programmer means external to the body for programming said stimulation means;
whereby, said pulsed electrical stimulation to said vagus nerve leads to increased cardiac parasympathetic tone.

19. The method of claim 18, wherein said stimulation means comprises an implantable pulse generator with fixed programs which is controllable with an external magnet.

20. The method of claim 18, wherein said programmer means are remotely operated via the internet.

21. The method of claim 18, wherein said stimulation means can be remotely controlled over a wireless wide area network.

22. The method of claim 18, wherein said implantable components comprise an implantable pulse generator (IPG) with a recharging coil for recharging the implantable pulse generator using an external power source.

23. A system of providing electrical pulses to one or both vagus nerve(s) and its branches of a patient, with a combination of implanted and external components to provide therapy for at least one of atrial fibrillation, congestive heart failure, inappropriate sinus tachycardia, and refractory hypertension, comprising:
a) a stimulation means; wherein said stimulation means comprising implantable and external components;
b) programming means; wherein said programming means comprising means for networking with remote computers for data exchange; and
c) programming said stimulation means with said programming means;
whereby, said electrical pulse therapy is provided as programmed.

24. The system of claim 23, wherein said stimulation means comprises an implantable pulse generator with at least two fixed programs which are activated with a magnet.

25. The system of claim 23, wherein said external component is a magnet.

26. The system of claim 23, wherein said external component is an external stimulator.

27. The system of claim 26, wherein said external stimulator further comprises telemetry means for networking.

28. The system of claim 23, wherein said programmer means can be remotely operated over a wide area network.

29. The system of claim 23, wherein said implantable components comprise an implantable pulse generator (IPG) with a recharging coil for recharging the implantable pulse generator using an external power source.

30. The system of claim 23, wherein said stimulation means comprises,
a) an implanted stimulus-receiver; said stimulus-receiver comprising circuitry and a high-value capacitor for storing charge; and
b) an external stimulator for delivering power and data.
